(19) United States
(12) Patent Application Publication
(10) Pub. No.: US 2013/0029378 A1
(43) Pub. Date: Jan. 31, 2013

(54) NOVEL ENZYME

(71) Applicants: Benjamin Guy Davis, Oxfordshire (GB); Ayhan Celik, Oxfordshire (GB); Gideon John Davies, Yorkshire (GB); Karen Mary Ruane, West Midlands (GB)

(72) Inventors: Benjamin Guy Davis, Oxfordshire (GB); Ayhan Celik, Oxfordshire (GB); Gideon John Davies, Yorkshire (GB); Karen Mary Ruane, West Midlands (GB)

(73) Assignee: ISIS INNOVATION LIMITED, Oxford, OX (GB)

(21) Appl. No.: 13/122,954

(22) PCT Filed: Oct. 6, 2009
(86) PCT No.: PCT/GB2009/051320

§ 371 Date: Feb. 8, 2012

Related U.S. Application Data
(63) Continuation of application No. 12/343,897, filed on Dec. 24, 2008, now abandoned.

(30) Foreign Application Priority Data
Oct. 7, 2008 (GB) 0818328.7

(51) Int. Cl. C12N 9/06; C12N 15/53; C12N 1/21; C12P 19/36; C12N 15/63

(52) U.S. Cl. 435/90; 435/191; 435/320.1; 435/252.33; 536/23.2

(57) ABSTRACT

The invention provides an isolated formate dehydrogenase (FDH) polypeptide specific for NADP⁺ and an isolated FDH polypeptide having an adenine ribose recognition loop comprising a first large amino acid and a second amino acid, wherein the first and second amino acid are arranged in space to allow the second amino acid to bond with a phosphate group. Also provided is a variant of an BAD⁺ specific FDH polypeptide, wherein the adenine ribose recognition loop has been mutated at least one position to alter the three dimensional polypeptide structure of the adenine ribose recognition loop to allow a phosphate group to be recognised. The polypeptides of the invention can be used in the conversion of NADP⁺ to NADP or in the conversion of BAD⁺ to NASH.

The present invention provides novel FDH enzymes and in particular novel NADPH-specific or NADH-specific FDH enzymes. The invention also provides the use of these novel enzymes in catalytic systems for the in situ regeneration of NADPH or NADH. The invention also relates to the crystal structure of the novel enzymes and the use of these structures.

The ability of enzymes to operate simply in aqueous systems in a highly efficient manner makes them attractive environmentally benign synthetic reagents. However, many classes of biocatalysts are not fully exploited, and their use in the large-scale enzymatic synthesis of high added-value chemicals is often limited, by the need for expensive co-factors. Oxidoreductases represent some 25% of all known enzymes, and the vast majority are dependent on one of the two nicotinamide cofactors NADH or NADPH. (Liu, W. et al., Biotechnology Advances 25, 369-384 (2007)).

Formate dehydrogenase (FDH) enzymes allow the in situ regeneration of the redox co-factor NADH. FDH is therefore of considerable commercial interest as a catalyst for the regeneration of the reduced cofactor in the synthesis and/or biotransformation of valuable compounds. FDH-mediated NAD⁺ to NADH regeneration is regarded as the “gold standard” in cofactor regeneration (Liu, W. et al., (2007)), and has allowed the efficient exploitation of NAD(H)⁺-dependent oxidoreductases across a vast landscape of chemical syntheses. High profile industrial examples of FDH-mediated NAD⁺ to NADH regeneration include the production of tert-L-leucine and other non-proteinogenic amino acids, which may be useful in the production of pharmaceuticals.

All known wild type formate dehydrogenases are NADH-specific, exhibiting a strong preference for NADH over NADPH, and showing poor or no ability to catalyse the reaction with NADP⁺ (Seelbach, K. et al. Tetrahedron Letters 37, 1377-80 (1996); Tishkov, V. I. et al., Biotechnology and Bioengineering 64, 187-194 (1999)).

The absence of a comparable method for efficient NADP⁺ to NADPH cofactor regeneration radically impairs efficient “green” chemical synthesis, since over 80% of biocatalytic reductions utilize NADPH, not NADH. These processes involve a spectrum of over 300 known, repeatedly-used, reaction types (Woodyer, R., et al., FEBS Journal 272, 3816-3827 (2005)). Given the lack of a process for in situ NADPH regeneration, the discovery of an FDH with a preference for NADPH would release a valuable untapped chemical resource.

Despite a broad-ranging fundamental interest in dehydrogenase specificity (Hall, N. et al., Microbiology 146, 1399-1406 (2000); (Lamzin, V. S. et al., Journal of Molecular Biology 236, 759-785 (1994); Lamzin, V. S. et al., Current Opinion in Structural Biology 5, 830-836 (1995)), no FDH with a preference for NADPH is known.

According to a first aspect, the invention provides an isolated formate dehydrogenase (FDH) polypeptide which is NADPH-specific.

The terms NADPH-specific and NADP⁺-specific are used interchangeably herein and refer to FDH enzymes which catalyse the conversion of NADP⁺ to NADPH. Similarly, the terms NAD⁺ H-specific and NAD⁺-specific are used interchangeably herein and refer to FDH enzymes which catalyse the conversion of NAD⁺ to NADH.

An FDH polypeptide is defined as NADPH-specific if its ability to regenerate NADPH from NADP⁺ is greater than its ability to regenerate NADH from NAD⁺. The FDH polypeptide may be able to regenerate both NADPH and NADH, but to be specific for one it has to have an improved ability to regenerate that one. Preferably an NADP⁺-specific FDH polypeptide according to the invention displays a preference in favour of NADP⁺ that is more than 10⁶ times greater than those of known NAD⁺-specific FDHs. Preferably, an FDH protein according to the invention which is specific for NADPH has a preference for NADP⁺ over NAD⁺ of greater than 10 fold based on (kcat/Km)^{NADP+}/(kcat/Km)^{NAD+}, preferably greater than 20 fold, preferably greater than 25 fold, more preferably greater than 30 fold.

According to another aspect, the invention provides an isolated FDH polypeptide wherein the adenine ribose recognition loop comprises a first large amino acid and a second amino acid, wherein the first and second amino are arranged in space to allow the second amino acid to bond with a phosphate group.

Preferably the phosphate is part of NADP⁺. Preferably the polypeptide is able to recognize NADP⁺ and catalyse its conversion to NADPH. The polypeptide may also be able to catalyse the conversion of NAD⁺ to NADH. Preferably the polypeptide has a preference for NADP⁺ over NAD⁺.

Preferably the first large amino acid is an amino acid with a van der Waals volume of about 110 Anĝ3 or more. The first large amino acid may be selected from the group comprising glutamine, tyrosine, phenylalanine, methionine, isoleucine and leucine. The large amino acid may also be selected from arginine, histidine, lysine and tryptophan.

Preferably the second amino acid is able to form a hydrogen bond or an ionic bond, or both, with the phosphate. Preferably the second amino acid has a positive charge. The second amino acid may be selected from the group comprising arginine, lysine, glutamic acid, glutamine and aspartic acid.

Preferably the first large amino acid is glutamine or tyrosine. Preferably the second amino acid is arginine or lysine. Preferably the first amino acid is glutamine and the second amino acid is arginine.

Preferably the first amino acid and the second amino acid are no more than about 20 amino acids apart in the primary amino acid sequence of the FDH polypeptide. Preferably the first amino acid and the second amino acid are no more than 10 amino acids apart in the primary amino acid sequence of the FDH polypeptide. The first amino acid and the second amino acid may be adjacent in the primary amino acid sequence of the FDH polypeptide.

Preferably the first amino acid and the second amino acid are no more than about 10 angstroms apart in the folded FDH polypeptide. Preferably the first amino acid and the second amino acid are no more than about 9, 8, 7, 6, 5, 4, 3 or 2 angstroms apart; preferably the first amino acid and the second amino acid are no more than about 4 angstroms apart in the folded FDH polypeptide.

Preferably the first and second amino acids result in the adenosine ribose recognition loop being configured in the folded protein to accommodate and bond with the phosphate of NADP⁺, this is in contrast to known NAD⁺ specific FDH enzymes in which the structure of the adenine ribose recognition loop prevents recognition of NADP⁺.

The adenine ribose recognition loop is preferably less the 20 amino acids, preferably less than 15 amino acids, preferably less than 10 amino acids. The adenine ribose recognition loop in the novel FDH enzymes preferably comprises amino acids 222 to 228 in Seq ID No: 1 or 2, and more preferably comprises amino acids 222 to 227. The skilled man would be readily able to identify the adenine ribose recognition loop in other FDH enzymes based on primary amino acid sequence homology and/or three dimensional structure homology.

According to a further aspect, the invention provides an isolated polypeptide comprising an adenine ribose recognition loop wherein the amino acid sequence of the adenine ribose recognition loop has a least 50% or more sequence identity to the sequence of the adenine ribose recognition loop in Seq ID No: 1 or Seq ID No: 2.

Preferably the adenine ribose recognition loop of the polypeptide has at least about 60%, 70%, 80%, 90%, 95%, 98% or more sequence identity with the adenine ribose recognition loop in Seq ID No: 1 or Seq ID No: 2. Preferably the polypeptide has an adenine ribose recognition loop identical to that of Seq ID No: 1 or Seq ID No: 2.

The adenine ribose recognition loop preferably comprises amino acids 222 to 228 in Seq ID No: 1 or 2, and more preferably the adenine ribose recognition loop comprises amino acids 222 to 227 in Seq ID No: 1 or 2.

Preferably the polypeptide is a FDH enzyme.

Percentage sequence identity is defined as the percentage of amino acids in a sequence that are identical with the amino acids in a provided sequence after aligning the sequences and introducing gaps if necessary to achieve the maximum percent sequence identity. Alignment for purpose of determining percent sequence identity can be achieved in many ways that are well known to the man skilled in the art, and include, for example, using BLAST (National Center for Biotechnology Information Basic Local Alignment Search Tool). Variations in percent identity may be due, for example, to amino acid substitutions, insertions or deletions. Amino acid substitutions may be conservative in nature, in that the substituted amino acid has similar structural and/or chemical properties, for example the substitution of leucine with isoleucine is a conservative substitution.

According to a yet further aspect, the invention provides an isolated polypeptide comprising an amino acid sequence which has a least 50% or more sequence identity with the sequence of Seq ID No: 1 or Seq ID No: 2.

Preferably the polypeptide has at least about 60%, 70%, 80%, 90%, 95%, 98% or more sequence identity with the sequence of Seq ID No: 1 or Seq ID No: 2. Preferably the polypeptide has at least about 80% sequence identity with the sequence of Seq ID No: 1 or Seq ID No: 2. Preferably the polypeptide has an amino acid sequence identical to that of Seq ID No: 1 or Seq ID No: 2.

Preferably the polypeptide is a FDH enzyme.

Preferably a polypeptide according to the invention comprises a large amino acid at the position corresponding to amino acid 223 in Seq ID No: 1 or Seq ID No: 2. Preferably the large amino acid is an amino acid with a van der Waals volume of about 110 Anĝ3 or more. The large amino acid may be selected from the group comprising glutamine, tyrosine, phenylalanine, methionine, isoleucine and leucine. The large amino acid may also be selected from arginine, histidine, lysine and tryptophan.

Preferably a polypeptide according to the invention comprises an amino acid at the position corresponding to amino acid 224 in Seq ID No: 1 or Seq ID No: 2 which is able to form a H bond and/or an ionic bond with a phosphate. Preferably the amino acid has a positive charge. The second amino acid may be selected from the group comprising arginine, lysine, glutamic acid, glutamine and aspartic acid.

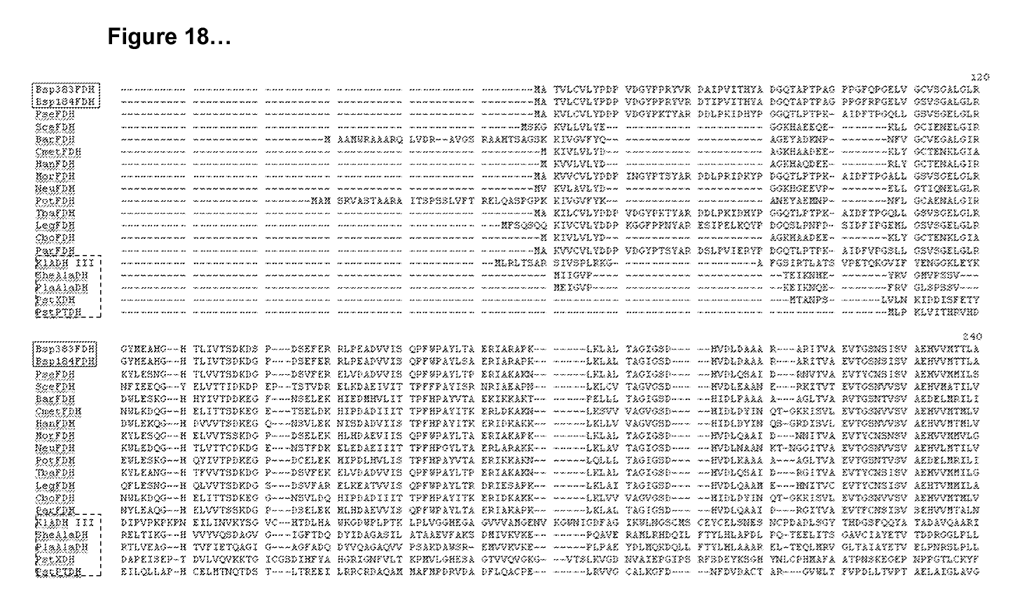

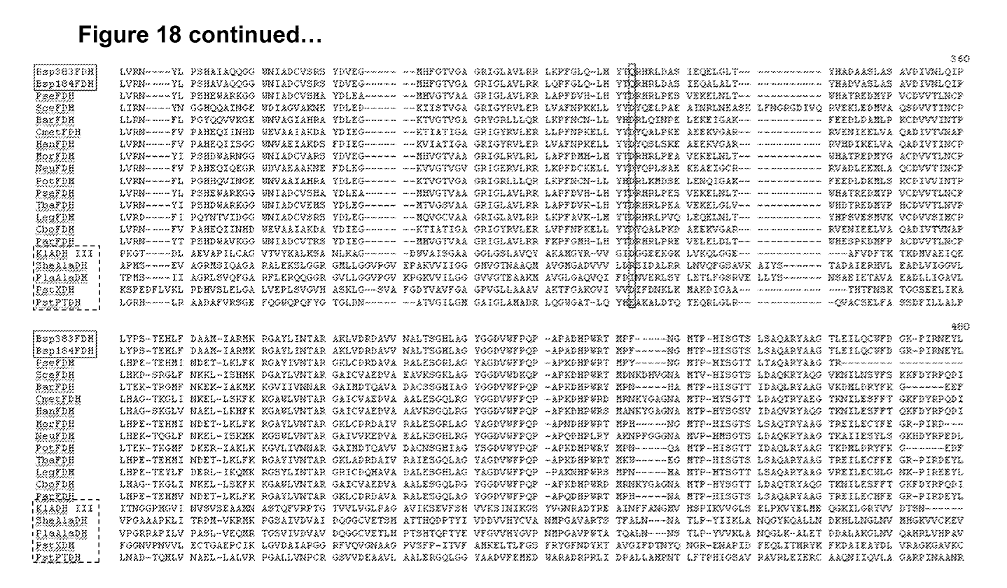

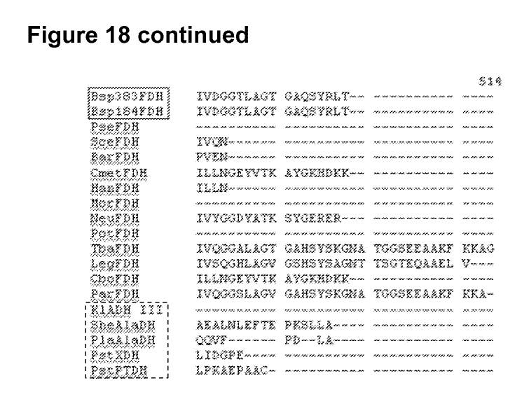

The amino acid corresponding to amino acid 223 or 224 in Seq ID No: 1 or Seq ID No: 2 could be readily determined in other FDH polypeptides by aligning the sequences; an example of such an alignment is given in FIG. 18.

A polypeptide according to the invention may have at least 50% sequence identity to the sequence of any of Seq ID Nos: 3 to 19. The polypeptide may have at least 60%, 70%, 80%, 90%, 95% or more sequence identity with the sequence of one or more Seq ID Nos: 3 to 19.

The polypeptide may be a naturally occurring polypeptide. Alternatively, the polypeptide may be a modified version of a naturally occurring polypeptide.

For example, the FDH protein may be a naturally occurring FDH enzyme from the Burkholderia sp. The FDH protein may be encoded by a gene derived from Burkholderia cenocepacia PC184. The protein may be referred to as BcenFDH1 or Bsp184FDH (Seq ID No: 2), and be encoded by the gene Bcenfdh1. Alternatively, the FDH protein may be encoded by a gene derived from Burkholderia sp 383. The protein may be referred to as BspFDH2 or Bsp383FDH (Seq ID No: 1), and be encoded by the gene Bspfdh2.

According to a further aspect, the invention provides a polynucleotide encoding a polypeptide of the invention. The polynucleotide may be included in a recombinant expression vector, wherein the polynucleotide may be operably linked to a promoter.

The invention may also provide a cell comprising a polynucleotide or expression vector according to the invention.

According to another aspect, the invention provides a variant of an NAD⁺-specific FDH polypeptide, wherein the amino acid in the adenine ribose recognition loop which corresponds to amino acid 223 in Seq ID No: 1 or 2, is a large amino acid and the polypeptide recognizes NADP⁺. Preferably the large amino acid is an amino acid with a van der Waals volume of about 110 Anĝ3 or more. The large amino acid may be selected from the group comprising glutamine, tyrosine, phenylalanine, methionine, isoleucine and leucine. The large amino acid may also be selected from arginine, histidine, lysine and tryptophan.

Preferably the variant polypeptide is a modified known FDH polypeptide.

Preferably the amino acid in the variant of an NAD⁺ specific FDH polypeptide which corresponds to amino acid 224 in Seq ID No: 1 or Seq ID No: 2 is able to form a H bond and/or an ionic bond with a phosphate. Preferably the amino acid has a positive charge. The amino acid may be selected from the group comprising arginine, lysine, glutamic acid, glutamine and aspartic acid.

Preferably the amino acid at the position corresponding to position 223 in Seq ID No: 1 or Seq ID No: 2 is glutamine and the amino acid at the position corresponding to position 224 in Seq ID No:1 or Seq ID No: 2 is arginine.

Preferably the variant of an NAD⁺ specific FDH polypeptide recognises NAD⁺ and NADP⁺. The variant may be NADP⁺ specific. Preferably the variant recognise NADP⁺ better than the unmodified NAD⁺ specific FDH polypeptide.

According to another aspect the invention provides a variant of an NAD⁺ specific FDH polypeptide, wherein the adenine ribose recognition loop has been mutated at least one position to alter the three dimensional polypeptide structure of the adenine ribose recognition loop to allow a phosphate group to be recognised. Preferably the variant of an NAD⁺ specific FDH polypeptide is able form a H bond and/or an ionic bond with a phosphate group.

Preferably the adenine ribose recognition loop comprises a first large amino acid and a second amino acid able to form a H bond and/or an ionic bond with a phosphate group. Preferably at least one or the first or second amino acids were not present in the unmutated NAD⁺ specific FDH polypeptide.

Preferably the first large amino acid is an amino acid with a van der Waals volume of about 110 Anĝ3 or more. The first large amino acid may be selected from the group comprising glutamine, tyrosine, phenylalanine, methionine, isoleucine and leucine. The large amino acid may also be selected from arginine, histidine, lysine and tryptophan.

Preferably the second amino acid is able to form a hydrogen bond or an ionic bond, or both, with the phosphate. Preferably the second amino acid has a positive charge. The second amino acid may be selected from the group comprising arginine, lysine, glutamic acid, glutamine and aspartic acid.

Preferably the variant polypeptide has an improved ability to catalyse the conversion of NADP⁺ to NADPH compared to the unmutated enzyme. Preferably the ability to catalyse the conversion of NADP⁺ to NADPH is improved by at least 10 fold, preferably at least 100 fold, preferably at least 1000 fold, compared to the unmutated polypeptide. Preferably the variant polypeptide is NADP⁺ specific.

According to another aspect, the invention provides a method of preparing an FDH polypeptide which recognizes NADP⁺ comprising:
a. providing a parent polypeptide having FDH activity capable of catalysing the conversion of NAD⁺ to NADH and having an amino acid sequence with at least 50% sequence identity to the sequence of one or more of Seq ID Nos: 3 to 19;
b. selecting an amino acid residue in the parent polypeptide at a position corresponding to amino acid 223 in Seq ID No: 1 or Seq ID No: 2;
c. providing an alternative amino acid at the position selected in b) to that which occurs in a), preferably the alternative amino acid is a large amino acid, preferably glutamine;
d. preparing a polypeptide with the sequence of c);
e. selecting a polypeptide prepared in d) which can recognize NADP⁺.

Preferably, in step (a) the polypeptide has at least 60%, 70%, 80%, 85%, 90%, 95% or more sequence identity to one or more of the sequences of Seq ID Nos: 3 to 19.

Preferably the polypeptide selected in (e) is at least 10 fold, preferably at least 100 fold, preferably at least 1000 fold, more efficient at catalysing the conversion of NADP⁺ to NADPH than the polypeptide in (a). Preferably the polypeptide in (e) is NADP⁺ specific.

According to another aspect, the invention provides a method of preparing an FDH polypeptide which recognizes NADP⁺ comprising:
a. providing a parent FDH polypeptide specific for NAD⁺, which preferably has an amino acid sequence with at least 50% sequence identity to the sequence of one or more of Seq ID Nos: 3 to 19;
b. identifying the adenine ribose recognition loop in the parent FDH polypeptide;
c. changing at least one amino acid residue in the adenine ribose recognition loop of the parent such that the loop can now recognise the phosphate of NADP⁺;
d. preparing a polypeptide with the sequence of c);
e. selecting a polypeptide prepared in d) which can recognize NADP⁺.

Preferably the polypeptide in (e) is able to catalyse the conversion of NADP⁺ to NADPH.

Preferably the polypeptide in (e) is at least 10 fold, preferably at least 100 fold, preferably at least 1000 fold, better at recognising NADP⁺ than the parent polypeptide. Preferably the polypeptide in (e) can recognise NAD⁺ and NADP⁺, and catalyse the conversion of each to NADH and NADPH respectively. Preferably the polypeptide in (e) is NADP⁺ specific.

Preferably in (c) the at least one amino acid introduced is a large amino acid. Preferably a large amino acid is an amino acid with a van der Waals volume of about 110 Anĝ3 or more. The large amino acid may be selected from the group comprising glutamine, tyrosine, phenylalanine, methionine, isoleucine and leucine. The large amino acid may also be selected from arginine, histidine, lysine and tryptophan. Preferably the large amino acid perturbs the 3D structure of the adenine ribose recognition loop in the parent FDH enzyme allowing the enzyme to recognise NADP⁺ and catalyse its conversion to NADPH.

According to yet another aspect, the invention provides a polypeptide produced by any method of the invention. Preferably the polypeptide produced is able to catalyse the conversion of NADP⁺ to NADPH.

Preferably the novel NADPH-specific formate dehydrogenase enzymes (FDHs) of the invention not only exhibit powerful formate dehydrogenase activity but also display a preference for the cofactor NADP⁺ over NAD⁺. From the crystal structures of these novel FDHs, a structural basis for formate dehydrogenase cofactor recognition has been determined for the first time. At a global structural level the novel NADP⁺-specific FDH proteins are similar to known NAD⁺-specific FDH enzymes and the majority of their interactions are the same as those observed for the NAD⁺-specific FDHs, but there are significant and key differences in their adenine-ribose recognition loops. Recognition of the phosphoriboside in the NADP⁺-specific FDH enzymes is conferred by a large amino acid, such as glutamine, in contrast to the smaller amino acid aspartic acid, found in the equivalent position of many known NAD⁺-specific FDH enzymes.

The novel cofactor preferences shown by FDHs according to the invention that are NADP⁺-specific, make in situ NADPH regeneration possible. Therefore, providing for the first time, efficient “green” chemical syntheses using enzymes that are NADPH dependent, this includes, but is not limited to, the synthesis of unnatural amino acids, chiral alcohols and polyols.

Both the novel FDH proteins, Seq ID No: 1 and 2, encoded by Burkholderia strains show powerful FDH activity, each having strong cofactor preferences for NADP⁺, (kcat/Km)^{NADP+}/(kcat/Km)^{NAD+} of 73 for BcenFDH1 and 39 for BspFDH2, some 10⁵ difference in NADP⁺ cofactor preference, and 10⁶ times greater than those of, e.g., CmetFDH (an FDH from Candida methylica).

Both BcenFDH1 (Bsp184FDH) and BspFDH2 (Bsp383FDH) have a glutamine residue at position 223 in the adenine-ribose recognition loop (see FIG. 18), which is involved in conferring recognition of NADP⁺. This is in contrast with NAD⁺-specific Burkholderia FDH enzymes which have an aspartate at the position which corresponds to position 223 in BcenFDH1 and BspFDH2. Gln223 is a key recognition element for the phosphate of NADP⁺ which makes BspFDH2 and BcenFDH1 specific for the phosphoribose of NADP⁺.

BcenFDH1 and BspFDH2 are examples of wild type FDHs that show a natural preference for NADP⁺ over NAD⁺. The importance of this invention is evident when it is considered that despite over 200 previous reported attempts to change cofactor preferences of NADH specific FDHs over the last three decades, only a few have shown any improvement and even then with only limited success (see Table 4).

In addition to naturally occurring FDH enzymes which have a glutamine in the adenine-ribose recognition loop, other FDH enzymes, such as those which are NAD⁺-specific, can be engineered/modified to be NADP⁺ specific. This may be achieved by introducing a large amino acid, such as glutamine, in the adenine-ribose recognition loop at the position corresponding to position 223 in BcenFDH1 and BspFDH2. Known NAD⁺-specific proteins have an aspartic acid in the adenine-ribose recognition loop at the position corresponding to position 223 in BcenFDH1 and BspFDH2. By changing the aspartic acid to a glutamine a dramatic shift in cofactor preference in favour of NADP⁺ and away from NAD⁺ is observed.

The glutamine, or other large amino acid, may be introduced by genetic engineering; that is by modifying the gene encoding the NAD⁺-specific FDH protein to introduce a glutamine, or by in vitro synthesis of the protein. In NAD⁺-specific FDH enzymes the aspartic acid residue in the adenine recognition loop, at the position corresponding to position 223 in BcenFDH1 and BspFDH2, binds the OH-3′ hydroxyl group of the adenine ribose moiety of NAD⁺. This residue is highly conserved in many NAD⁺-dependent FDHs containing Rossmann folds, and plays several critical roles in NAD⁺ specificity, viz. by hydrogen bonding to the ribose itself, by virtue of its hydrogen-bonding to either flank of the recognition loop, it defines the available environment for the adjacent arginine and provides potential electrostatic repulsion of the negatively charged phosphate group of NADP⁺. Replacement of the aspartic acid with a glutamine, or other large amino acid, in natural NAD(H) specific FDHs provides NADP(H) recognition with co-factor switching up to 10⁵-fold.

Currently available NAD⁺-specific FDHs have specificity ratios (kcat/Km)^{NADP+}/(kcat/Km)^{NAD+} in the range of 10³-10³⁰. However, as discussed above, the specificity of NAD⁺-specific FDHs can be changed by changing the aspartic acid in the adenine-ribose recognition loop, at the position corresponding to position 223 in BspFDH2 and BcenFDH1, to glutamine. For example, by changing the aspartic acid residue, Asp195, in the NAD⁺-specific wild type enzyme CmetFDH-wt to a glutamine (Asp195 is the amino acid in CmetFDH corresponding to Gln223 in BcenFDH1 and BspFDH2), a 5000-fold shift in cofactor preference from NAD⁺ to NADP⁺ is observed. Similarly, changing the glutamine at position 223 in BcenFDH1 (Bsp184FDH) and BspFDH2 (Bsp383FDH) to aspartic acid causes a similar cofactor preference shift, but this time from NADP⁺ in favour of NAD⁺. Thus, a glutamine either in a natural FDH enzyme or in an engineered NADH-specific enzyme provides NADP⁺ recognition with cofactor preferences switching up by 10⁵-fold.

According to another aspect, the invention provides an FDH polypeptide that has been engineered to alter cofactor preference between NADP⁺ and NAD⁺. For example, the FDH polypeptide may naturally prefer NAD⁺ and may be engineered to prefer NADP⁺, or vice versa. The FDH polypeptide may be engineered by changing the amino acid, which may be an aspartic acid, at the position corresponding to position 223 in Seq ID No: 1 or 2 in the adenine-ribose recognition loop to a large amino acid, such as glutamine, or vice versa. In one embodiment, an FDH polypeptide which in the naturally occurring form has an aspartic acid in the adenine-ribose recognition loop at the position corresponding to position 223 in Seq ID No: 1 or 2, and therefore has a preference for NAD⁺, may engineered to have a glutamine at this position, and therefore is able to recognise NADP⁺ and preferably be specific for NADP⁺. Similarly, an FDH protein which in the naturally occurring form has a glutamine in the adenine-ribose recognition loop at the position corresponding to position 223 in Seq ID No: 1 or 2, and therefore has a preference for NADP⁺, may be engineered to have an aspartic acid at this position, and therefore recognise NAD⁺, and preferably be specific for NAD⁺.

According to another aspect, the invention provides a method of engineering the specificity of an FDH polypeptide by controlling the amino acid incorporated into the adenine-recognition loop at the position corresponding to position 223 in Seq ID No: 1 or 2.

FDH polypeptides according to the invention may have dual cofactor specificity; such polypeptides may have use in the industrial-level synthesis of a diverse range of products.

According to a further aspect, the invention provides the use of a polypeptide according to the invention in the conversion of NADP⁺ to NADPH or in the conversion of NAD⁺ to NADH.

According to a further aspect, the invention provides the use of a polypeptide according to the invention in an oxidoreductase process.

According to another aspect, the invention provides a method for the conversion of NADP⁺ to NADPH, or the conversion of NAD⁺ to NADH, comprising the steps of providing an FDH polypeptide according to the invention and adding it to NADP⁺ or NAD⁺.

According to another aspect, the invention provides an oxidoreductase process comprising the steps of providing an FDH polypeptide according to the invention and adding it to an oxidoreductase reaction mixture. Preferably, the FDH polypeptide converts NADP⁺ in the reaction mixture to NADPH and/or NAD⁺ in the reaction mixture to NADH.

It will be evident to the skilled man that the polypeptides of the invention may be used in a large number of oxido reduction reaction types utilising NADPH that were until now unattainable for efficiency, cost and waste stream reasons.

A polypeptide according to the invention may be used in an oxidoreductase process to regenerate NADH or NADPH. Preferably the polypeptide is used to regenerate NADPH.

The oxidoreductase process may cause oxygen C—H insertion, oxygen C—C insertion, hydride delivery or reductive amination.

The oxidoreductase process may comprise a monooxygenation reaction, a Baeyer-Villiger oxidation, a ketone reduction or D-amino acid synthesis.

More specifically, the oxidoreductase process may involve the highly efficient regio- and stereo-selective insertion of an oxygen atom into an inactivated C—H bond, for example, propylbenzene to obtain 1-phenyl-1-propanol. This may be successfully accomplished by coupling the enzymatic action of BspFDH2, or any other NADP⁺-specific FDH according to the invention, with that of cytochrome-P450-monooxygenase BM-3, in which one oxygen atom from an atmospheric O₂ molecule is reduced to water by NADPH, leaving the other oxygen atom ready for delivery to the substrate. All P450s are NADPH-dependent. The NADP⁺ thus formed is then returned to NADPH by the action of the FDH using formate. The overall atomic transformation is therefore a splitting of O₂, with a hydride ion being provided from formate and using NADP⁺ as a ‘hydride shuttle’. Few comparable chemical catalysts are known that directly oxidise such un-activated C—H bonds; none with such selectivity or efficiency.

The oxidoreductase process may involve biocatalytic Baeyer-Villiger synthesis of optically pure lactones by insertion of an oxygen atom into a C—C bond, in a suitable starting material. In particular, this may involve the biocatalytic Baeyer-Villiger (B-V) oxidation converting cyclohexanone to caprolactone. This may be accomplished by coupling the enzymatic action of BspFDH2, or any other NADP⁺-specific FDH according to the invention, with that of cyclohexanone monooxygenase (CHMO). Stereoselective B-V oxidations of cyclic ketones allows rapid access to chiral lactones as valuable intermediates in enantioselective synthesis. Biocatalysis offers a green alternative to organometallic catalysts, but, until now, has been often thwarted by the need for NADPH regeneration.

The oxidoreductase process may involve stereoselective synthesis of D-amino acids, such as stereoselective hydride delivery to ethyl 4-chloro-3-oxobutanoate to yield optically active D-(S)-4-chloro-3-hydroxybutanoate ethyl ester. This ester is a key chiral pharmaceutical intermediate used in the enantioselective synthesis of, for example, slagenin B and C and HMG-CoA reductase inhibitors. This may be accomplished by coupling the enzymatic action of BspFDH2, or any other NADP⁺-specific FDH according to the invention, with that of beta-ketoreductase (βKR) KRED101. βKR-mediated chemo, regio- and diastereo-selective reductions of ketones, ketoacids, and ketoesters to chiral alcohols allows the synthesis of key pharmaceuticals ranging from anti-depressants to adrenergic drugs.

The oxidoreductase process may involve ketoreductase-mediated synthesis, such as for an unnatural amino acid synthesis (a variant of the Degussa tert-leucine synthesis). In particular, stereoselective reductive amination of 2-oxooctanic acid to D-hexylglycine may be achieved by coupling the enzymatic action of BspFDH2, or any other NADP⁺-specific FDH according to the invention, with that of D-amino acid dehydrogenase (DAADH). D-amino acids have been used as key components in many biologically important compounds including antibiotics, fertility drugs, anticoagulants and pesticides. Ampicillin (containing D-phenylglycine) is currently produced on a scale of >5000 tons per year.

According to a yet further aspect, the invention provides a method of constructing a variant of a parent FDH enzyme, wherein the parent FDH is not Bsp383FDH, which variant has FDH activity and at least one altered property as compared to the parent FDH, the method comprising:

i) comparing the three dimensional structure of the parent FDH enzyme with that of Bsp383FDH;
(ii) identifying a part of the parent FDH enzyme that is different to Bsp383FDH and which from structural and functional considerations is contemplated to be responsible for differences in one or more properties of interest;
iii) modifying the part of the parent FDH identified in ii)

The method may also comprise the step of testing the variant FDH constructed in iii) to ensure the selected property has been altered. The property to be altered may be selected from the group comprising: enzyme specificity; enzyme stability, for example under different conditions such as pH, temperature or chemical environment; enzyme kinetic properties, such as pH or temperature dependent activity; protein expression properties; and crystallization properties.

The parent FDH may be modified in the adenine ribose recognition loop. This modification may have the effect of altering coenzyme specificity of the parent FDH. The adenine ribose recognition loop preferably comprises amino acids corresponding to amino acids 222 to 227 in Bsp383FDH. The modification in iii) may result in the parent FDH resembling Bsp383FDH at the site of modification. The modification may be accomplished by deleting, replacing, or inserting one or more amino acids into the parent FDH polypeptide.

According to another aspect, the invention provides the use of the three dimensional coordinates of Bsp383FDH to determine modifications to made to a parent FDH enzyme in order to alter one or more properties of the parent FDH. The invention also provides for modified proteins made as a result of this use of the three dimensional coordinates of Bsp383FDH.

The parent FDH may Bsp383FDH or another FDH enzyme. The properties to be altered may be selected from the group comprising changing enzyme specificity, improving thermal stability of the enzyme, improving enzyme stability in a particular environment, for example, at particular pH or in a particular aqueous or non-aqueous solvent, improving crystallization, improving kinetic properties of the enzyme and improving the level and/or rate of protein expression, for example in E. coli.

Enzyme specificity may be changed to alter co-factor specificity, for example from NAD⁺ to NADP⁺ or from NADP⁺ to NAD⁺. Alternatively, enzyme specificity may be changed to a different substrate.

This method of the invention may also be used to determine how to make fusion proteins involving the parent FDH enzyme.

The skilled man will appreciate that all preferred features discussed with reference to only some aspects of the invention can apply to all aspects of the invention.

The invention is illustrated by way of example only with reference to the following figures:

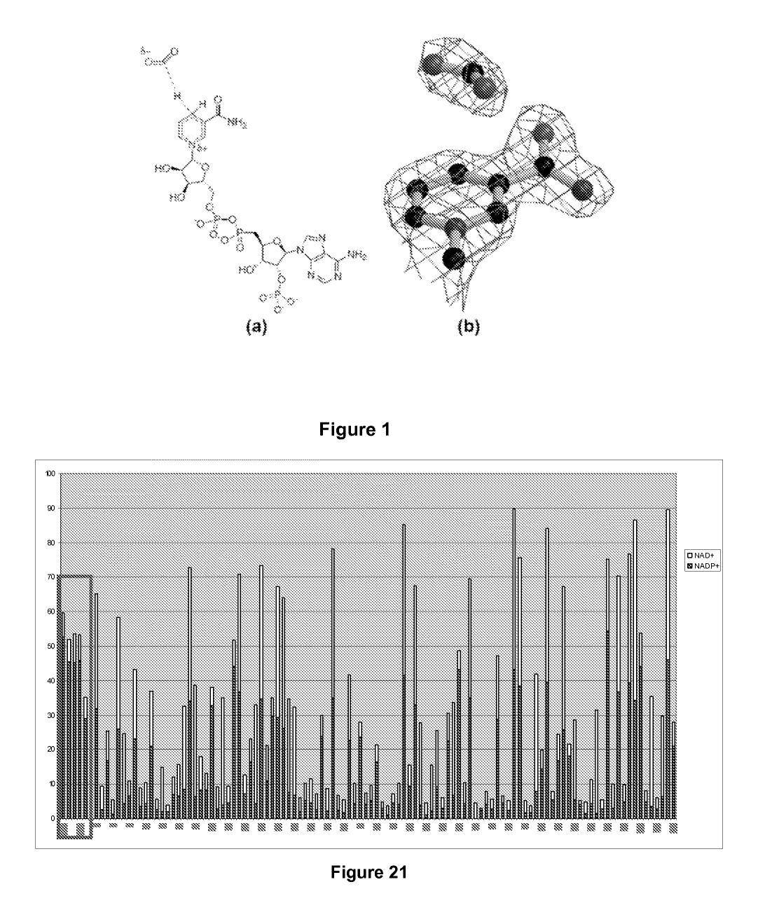

FIGS. 1 (a) and (b)—show the formate dehydrogenation reaction;

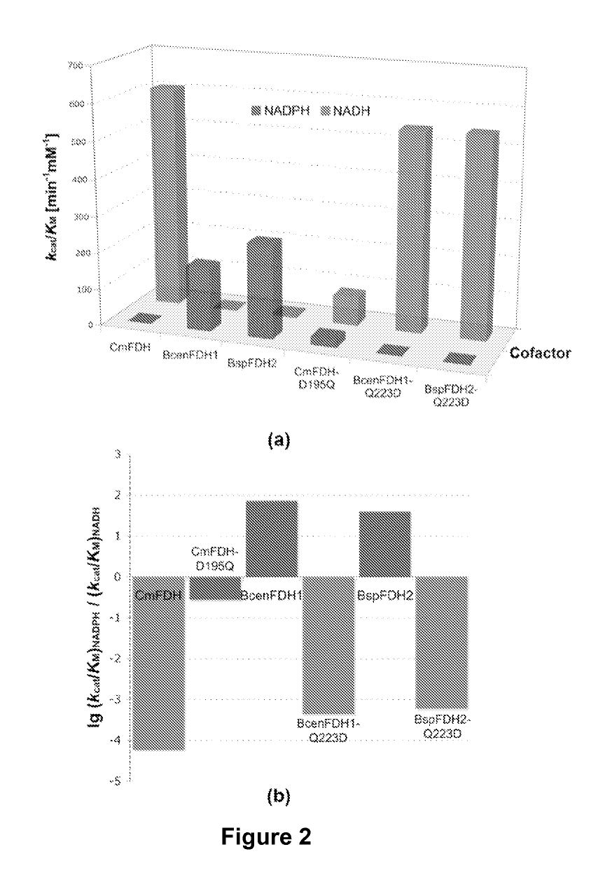

FIGS. 2 (a) and (b)—show kinetic activities and coenzyme preferences for various FDH enzymes including BcenFDH1 (42462 Da) and BspFDH2 (42466 Da);

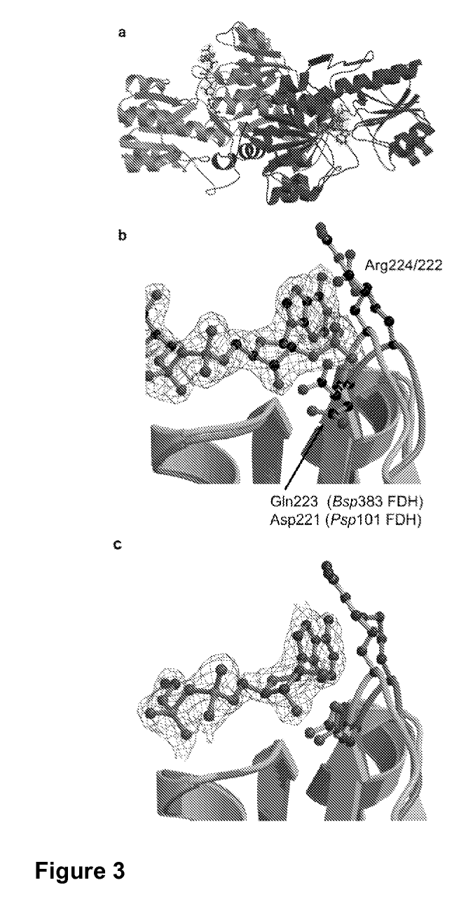

FIGS. 3 (a), (b) and (c)—show the 3D structural basis for cofactor recognition by the FDHs BspFDH2 and Psp101FDH;

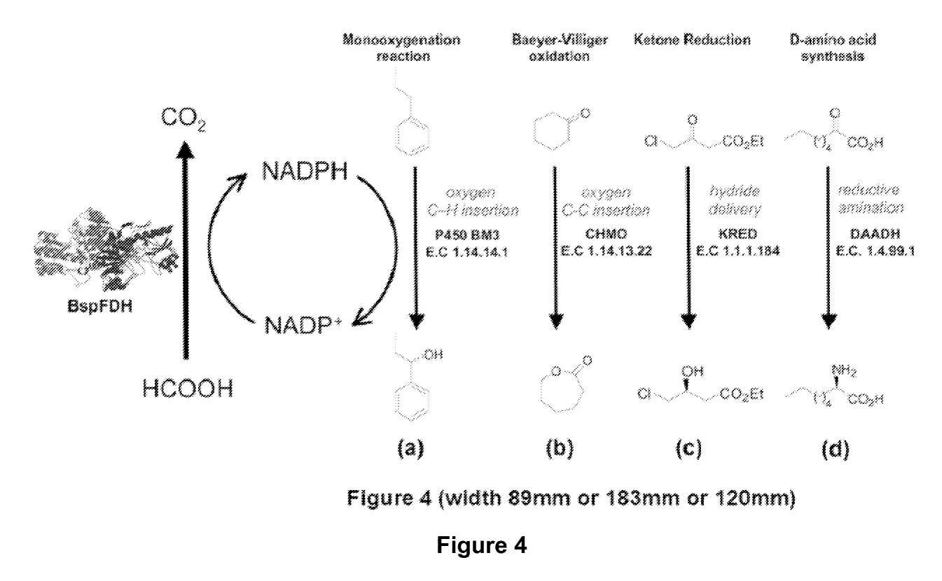

FIGS. 4 (a), (b), (c) and (d)—show biocatalytic applications using BspFDH coupled NADPH regeneration;

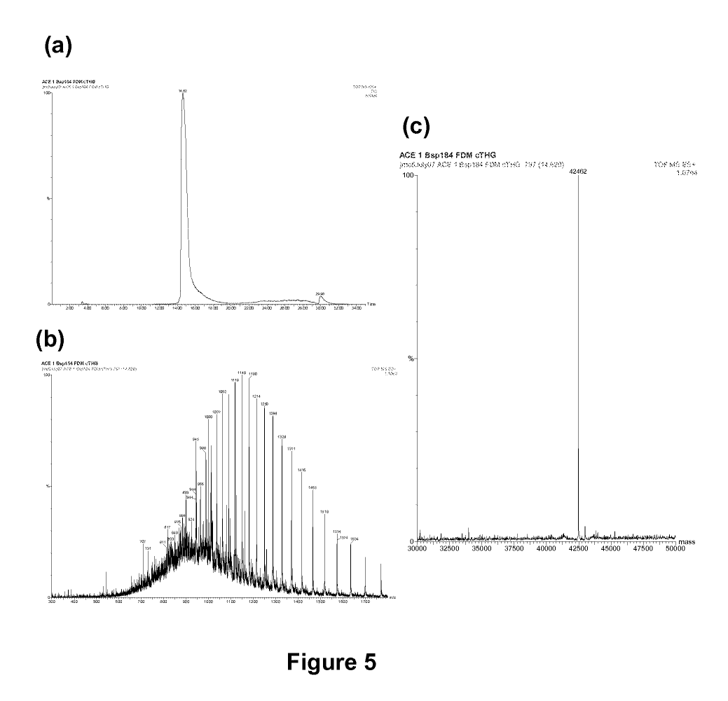

FIGS. 5 (a), (b) and (c)—shows the mass spectrometric analysis of BcenFDH1 (Bsp184FDH). FIG. 5(a) is a chromatogram, FIG. 5(b) is the multiple charge state “RAW” spectrum, and FIG. 5(c) is the MaxEnt deconvoluted spectrum;

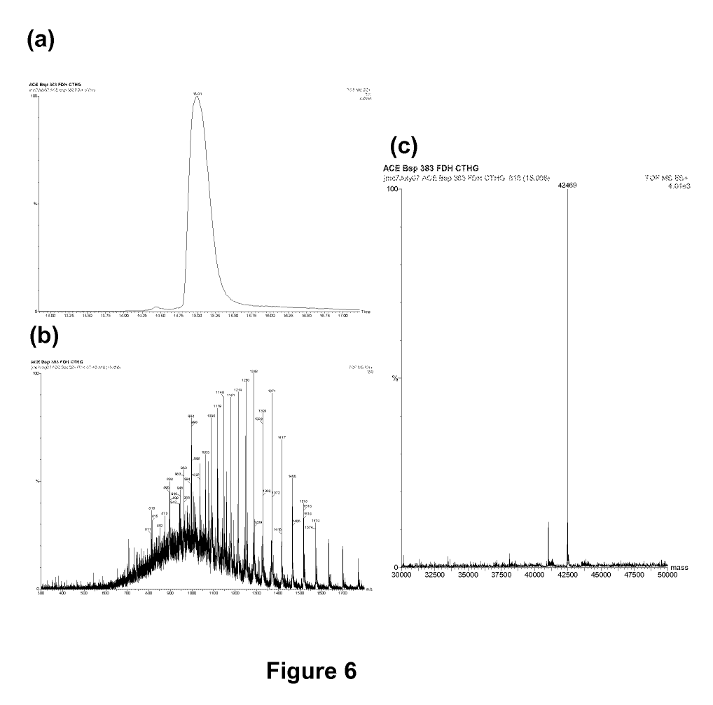

FIGS. 6 (a), (b) and (c)—shows the mass spectrometric analysis of BspFDH2 (Bsp383FDH). FIG. 6(a) is a chromatogram, FIG. 6(b) is the multiple charge state “RAW” spectrum, and FIG. 6(c) is the MaxEnt deconvoluted spectrum;

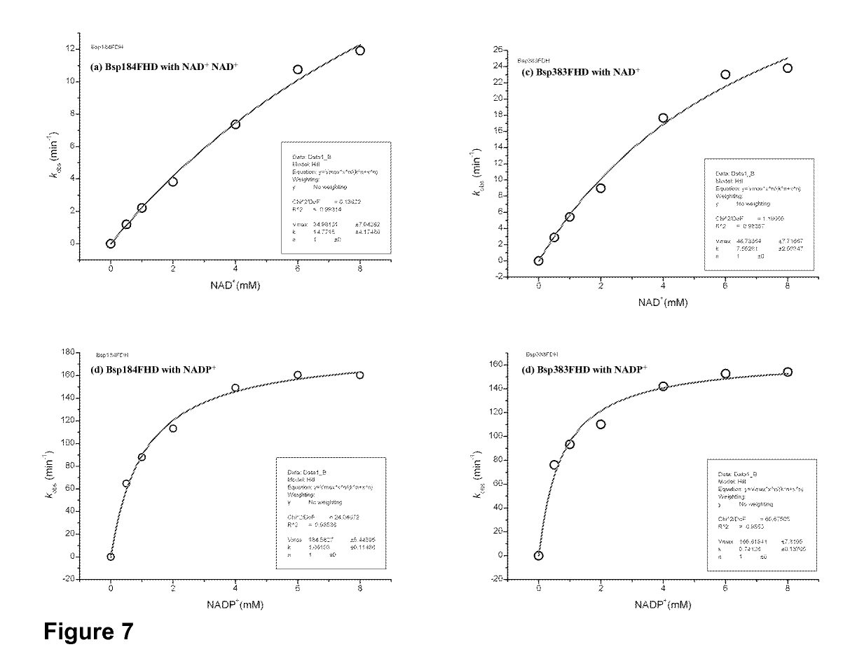

FIGS. 7 (a), (b), (c) and (d)—show the Michaelis-Menten kinetics for cofactor preference studies catalyzed by Bsp184FDH (a) with NAD⁺ and (b) with NADP⁺ and; by Bsp383FDH (c) with NAD⁺ and (d) with NADP⁺.

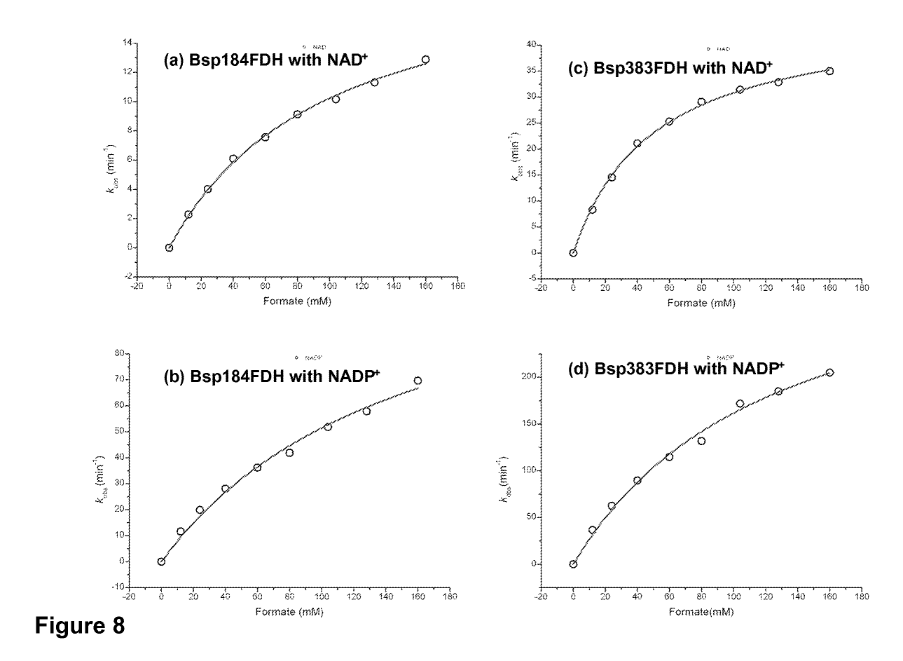

FIGS. 8 (a), (b), (c) and (d)—show the Michaelis-Menten kinetics for formate oxidation catalyzed by Bsp184FDH (a) with NAD⁺ and (b) with NADP⁺ and, by Bsp383FDH (c) with NAD⁺ and (d) with NADP⁺.

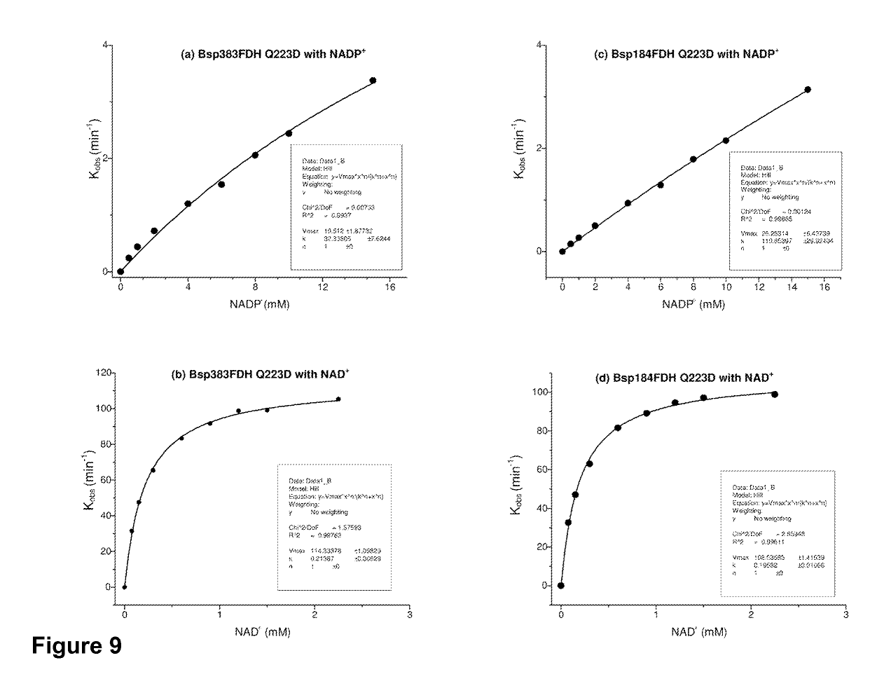

FIGS. 9 (a), (b), (c) and (d)—show the Michaelis-Menten kinetics for cofactor preference studies catalyzed by Q223D Bsp383FDH mutant (a) with NADP⁺ and (b) with NAD⁺ and; by Q223D Bsp184FDH mutant (c) with NADP⁺ and (d) with NAD⁺.

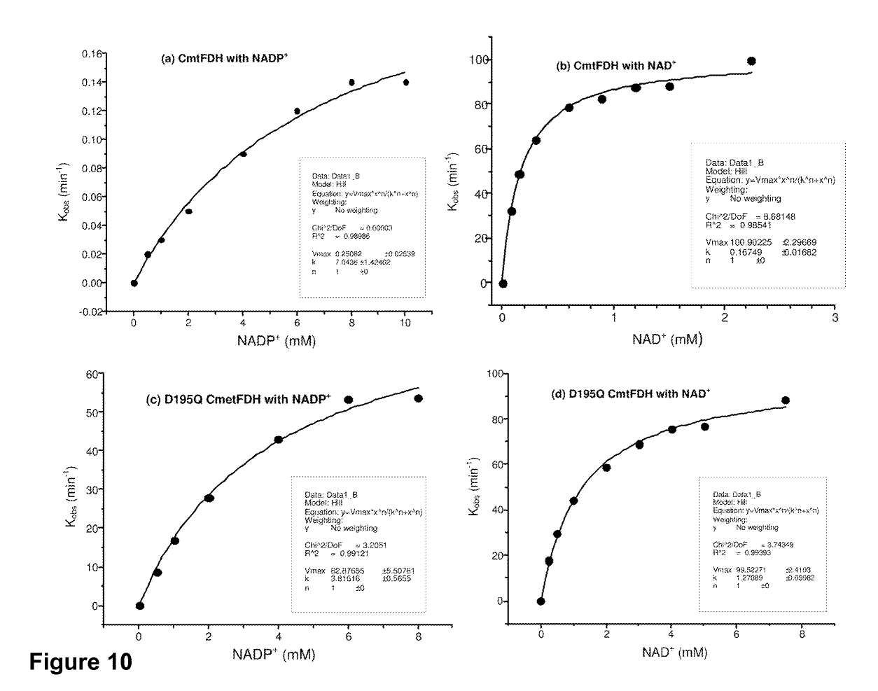

FIGS. 10 (a), (b), (c) and (d)—show the Michaelis-Menten kinetics for cofactor preference studies catalyzed by CmetFDH (a) with NADP⁺ and (b) with NAD⁺ and; by D195Q CmetFDH mutant (c) with NADP⁺ and (d) with NAD⁺.

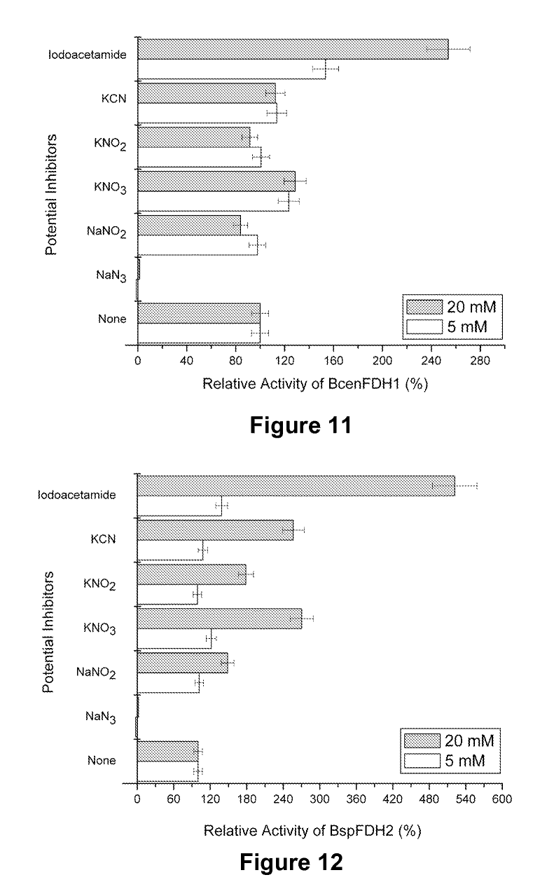

FIG. 11—shows the inhibition effect of several anions on the % relative activity of Bsp184FDH(BcenFDH1).

FIG. 12—shows the inhibition effect of several anions on the relative activity of Bsp383FDH(BspFDH2).

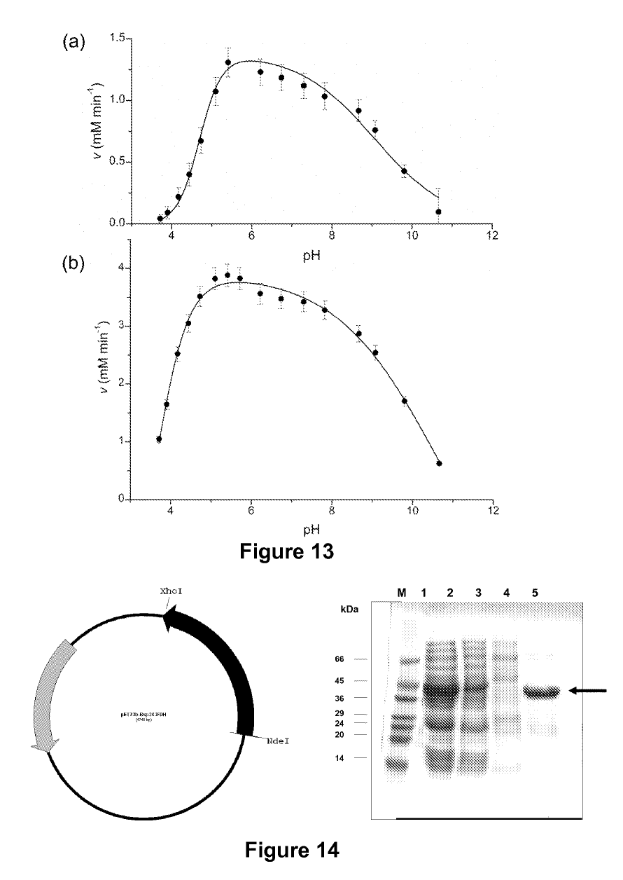

FIGS. 13 (a) and (b)—demonstrate the operative pH range of the purified (a) BcenFDH1 and (b) BspFDH2 enzymes.

FIG. 14—illustrates the construction of the expression vector pET23b-Bsp383FDH and the SDS-PAGE analysis of the expressed protein Bsp383FDH and purification of Bsp383FDH from an E. coli culture of BL21(DE3)plysS containing the pET23b-Bsp383FDH plasmid following induction with IPTG.

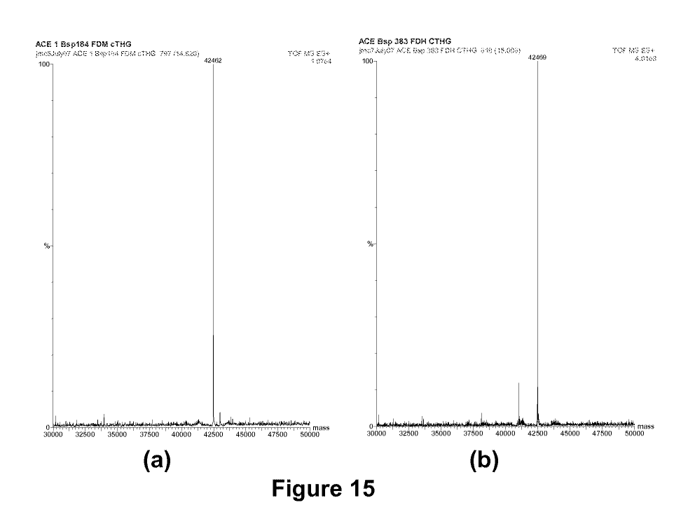

FIGS. 15 (a) and (b)—shows the LCMS analysis of (a) BcenFDH1 and (b) BspFDH2.

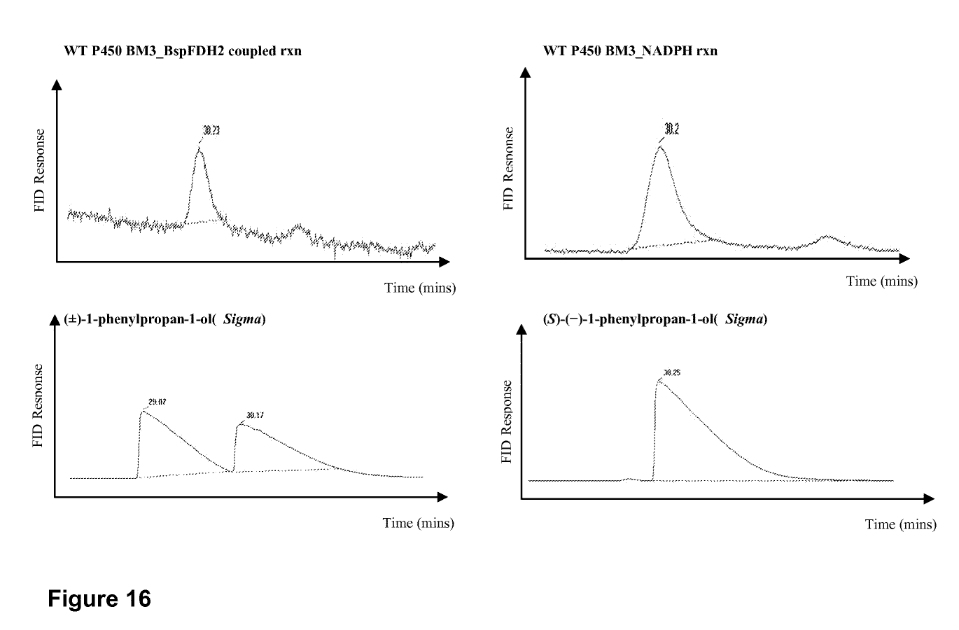

FIG. 16—shows gas chromatograms of 1-propylbenzene oxidation by wild type and mutant P450 BM3 in an NADPH regeneration reaction: cytochrome P450 BM3/BspFDH2 coupled oxidation of 1-propylbenzene.

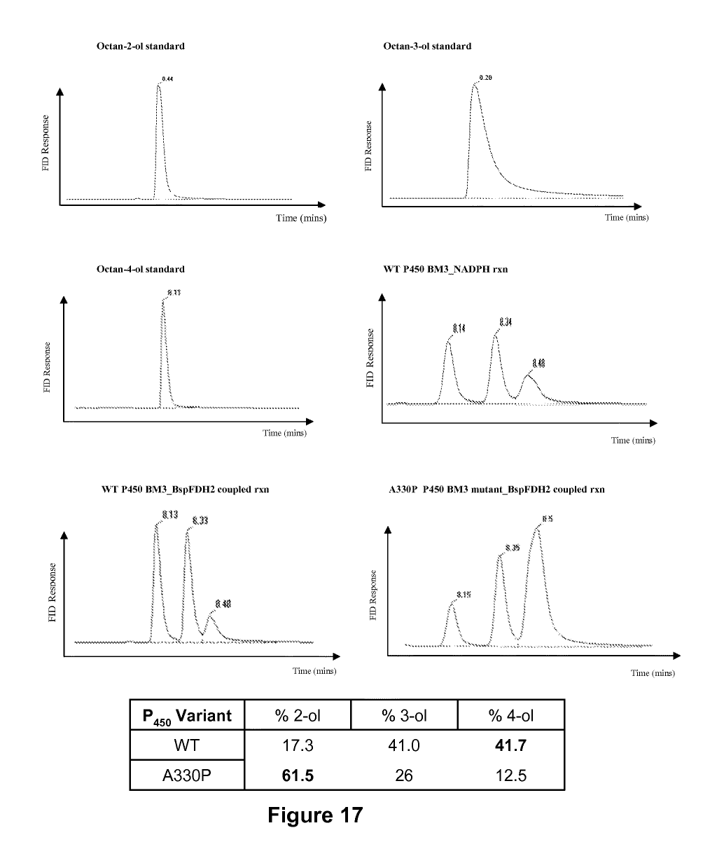

FIG. 17—shows gas chromatograms of octane oxidation by wild type and mutant P450 BM3 in NADPH regeneration reaction: cytochrome P450 BM3/BspFDH2 coupled oxidation of octane.

FIG. 18—shows the protein sequence alignment of a number of FDH proteins. More specifically of:

Burkholderia sp. 383 (Bsp383FDH, NCBI YP_{—}366697)—Seq ID No: 1:

Burkholderia cenocepacia PC184 (Bsp184FDH, NCBI EAY67119)—Seq ID No: 2:

Pseudomonas sp. 101 (Pse FDH, NCBI FDH_PSESR)—Seq ID No: 3:

S. cerevisiae (SceFDH, EMBL Z75296)—Seq ID No: 4:

barley (BarFDH, EMBL D88272)—Seq ID No: 5:

Candida methylica (CmetFDH, EMBL X81129)—Seq ID No: 6:

Hansenula polymorpha (HanFDH, EMBL P33677)—Seq ID No: 7:

Moraxella sp. C-1 (MorFDH, EMBL Y13245)—Seq ID No: 8:

Neurospora crassa (NeuFDH, EMBL L13964)—Seq ID No: 9:

potato (PotFDH, EMBL Z21493)—Seq ID No: 10:

Thiobacillus sp. KNK65MA (TbaFDH, NCBI BAC92737)—Seq ID No: 11:

Legionella pneumophila (LegFDH, NCBI AAU26390)—Seq ID No: 12:

Candida Boidinii (CboFDH, NCBI CAA09466)—Seq ID No: 13:

Paracoccus sp. 12-A (ParFDH, NCBI BAB64941)—Seq ID No: 14:

Dehydrogenase from Kluyveromyces lactis (KIADH III, NCBI P49384)—Seq ID No: 15:

Dehydrogenase from Shewanella sp. Ac10 (SheAlaDH, NCBI AAC23578)—Seq ID No: 16:

Dehydrogenase from Phormidium lapideum (PlaAlaDH, NCBI BAA24455)—Seq ID No: 17:

Dehydrogenase from Pichia stipitis (PstXDH, NCBI CAA39066)—Seq ID No: 18

Dehydrogenase from Pseudomonas stutzeri (PstPTDH, NCBI O69054)—Seq ID No: 19.

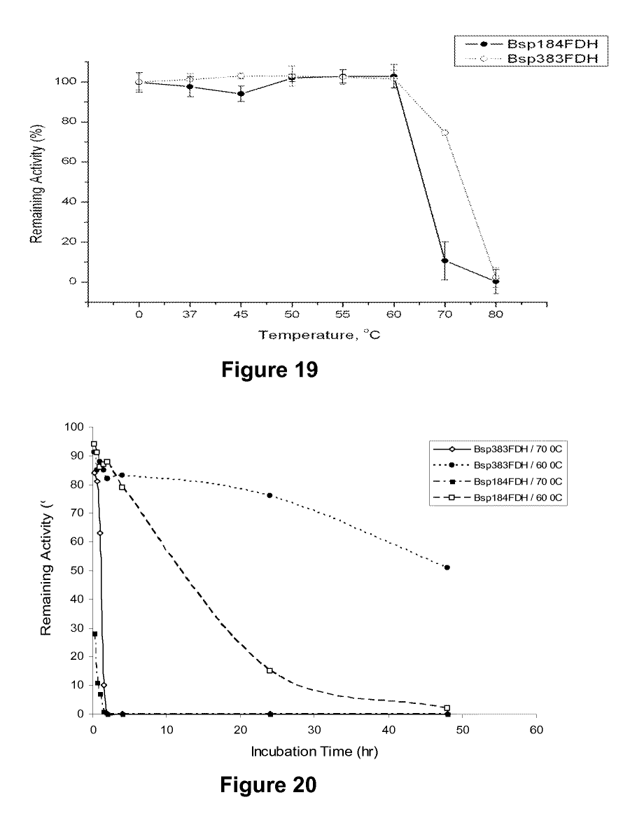

FIGS. 19 and 20—show the effect of temperature on the stability of the Bsp184FDH and Bsp383FDH enzymes. In FIG. 19 Bsp184FDH (2.1 mg/mL) and Bsp383FDH (1.5 mg/mL) were incubated at different temperatures for 20 minutes in 20 mM Tris-HCl buffer pH 7.2 and then stored at 0° C. until use. Remaining activities were assayed under standard assay conditions and were expressed as the percentage of activities. In FIG. 20 Bsp184FDH (2.1 mg/mL) and Bsp383FDH (1.5 mg/mL) were incubated at 60° C. and 70° C. for a 48 hour period in 20 mM Tris-HCl buffer pH 7.2 and then stored at 0° C. until use. Remaining activities were assayed under standard assay conditions and were expressed as the percentage of activities.

FIG. 21—shows the activity data from screening for Q223X of 103 colonies in wells. The x axis shows the well numbers and the y axis shows the activity levels. NAD⁺ activity is shown in the light coloured bars and NADP⁺ activity is shown in the dark coloured bars. Wells 1A-1H (shown in the boxed area) correspond to wild type (WT) activity; wells 9-103 correspond to tested random colonies.

The terms Bsp184FDH and BcenFDH1 refer to the same FDH enzyme and are used interchangeably herein. Similarly, the terms Bsp383FDH and BspFDH2 refer to the same FDH enzyme and are used interchangeably herein.

Formate dehydrogenase catalyses the oxidation of formate ion into CO, and hydride H. Formate dehydrogenases that are NADH-specific share high (>40%) amino acid sequence similarity and, where known, share similar 3-D structures (Lamzin, V. S. et al., Journal of Molecular Biology 236, 759-785 (1994)) with apparently identical catalytic sites for hydride-transfer.

Known NAD⁺ specific FDH proteins are homodimeric, each monomer consisting of cofactor and substrate binding domains, with hydride transfer occurring at the interface. The reaction they catalyze involves direct hydride transfer from substrate to cofactor by the cleavage of a carbon-hydrogen bond in the substrate and formation of carbon-hydrogen in the cofactor without proton release or abstraction. In this way, hydride H⁺ is efficiently trapped by NAD⁺ to form NADH, releasing CO₂ as the only, and easily managed, by-product. FIG. 1 shows formate reduction by FDH. FIG. 1(a) shows the transition-state of hydride transfer to/from CO₂/formate, FIG. 1(b) shows the 3D active site region structure in which hydride transfer takes place with the nicotinamide ring aligned over the formate substrate.

In striking contrast to all known formate dehydrogenases, the two novel proteins, BcenFDH1 (SEQ ID NO: 2) and BspFDH2 (SEQ ID NO: 1) have not only been shown to have powerful FDH activity, but also to display coenzyme preferences that lie strongly in favour of NADP⁺, 10⁶ times greater than those of known FDHs such as CmetFDH, a formate dehydrogenase from Candida methylica.

The BcenFDH1 and BspFDH2 proteins are encoded by the Burkholderia sp. FDH genes, Bcenfdh1 and Bspfdh2 (from Burkholderia cenocepacia PC184 FDH and Burkholderia sp. 383, respectively). The Bcenfdh1 and Bspfdh2 genes were amplified from genomic DNA and expressed in standard E. coli expression systems as their his-tagged forms (see Methods below) at levels around 20 mg/L.

The gene products were characterised including sequencing and protein mass spectrometry (ESI+: BcenFDH1 42462 Da calculated, 42462 Da found; BspFDH2 42466 Da calculated, 42469 Da found) which confirmed their structural identity. The protein sequence is given in FIG. 18.

Catalytic activity assessments (activities and coenzyme preferences) for the BcenFDH1 and BspFDH2 proteins, together with a representative example of previously known FDHs, were performed (see FIG. 2). These confirmed that currently available FDHs were highly NAD⁺-specific with specificity ratios, (kcat/Km)NADP⁺/(kcat/Km)NAD⁺, in the range of 10³-to-10¹⁰. For example, the widely-used (Tishkov, V. I., et al., Doklady Akademii Nauk SSSR 317, 745-8 [Biochem] (1991)) FDH from Pseudomonas sp. 101 (PseFDH) catalyses formate oxidation 2400 times more effectively with NAD⁺ than with NADP⁺ rendering the miniscule rate with NADP⁺ inappropriate for co-factor regeneration.

The FDH from Candida methylica (CmFDH) is 1.7×10⁴ times more effective with NAD⁺ than with NADP⁺ for formate oxidation (see Tables 1 and 2 showing cofactor preferences, respectively, for native Candida methylica (CmFDH) FDH, for BspFDH2, for BcenFDH1, for SceFDH and for PseFDH, together with the mutant FDH counterparts in which FDH specificity has been altered). Data has been included from Serov et al (Biochemical Journal 367 (3), 841-847 (2002)) and Andreadeli et al. (FEBS J. 275, 3859-3869 (2008)).

TABLE 1
Kinetic Parameters for Native FDHs

| | | | | NAD⁺ | | | |
|---|---|---|---|---|---|---|---|
| | NADP⁺ | | | | | | (k_{cat}/Kₘ₎^{NADP+}/ |
| Wild Types | Kₘ (mM) | k_{cat} (min⁻¹) | k_{cat}/Kₘ | Kₘ (mM) | k_{cat} (min⁻¹) | k_{cat}/Kₘ | ₍k_{cat}/Kₘ₎^{NAD+} |
| CmetFDH | 7.0 ± 1.4 | 0.30 ± 0.0 | 0.043 | 0.2 ± 0.0 | 100 ± 2 | 500 | 8.6 × 10⁻⁵ |
| | | | | | | | (0.18M formate) |
| Bsp184FDH | 1.1 ± 0.1 | 184 ± 5 | 167.2 | 15 ± 4 | 34 ± 7 | 2.27 | 73.66 |
| | | | | | | | (0.18M formate) |
| Bsp383FDH | 0.7 ± 0.1 | 166 ± 7 | 237.1 | 7.6 ± 2.1 | 49 ± 8 | 6.45 | 36.76 |
| | | | | | | | (0.18M formate) |
| | | | | | | | (k_{cat}/Kₘ₎^{NADP+}/ |
| | Kₘ (μM) | k_{cat} (s⁻¹) | k_{cat}/Kₘ | Kₘ (μM) | k_{cat} (s⁻¹) | k_{cat}/Kₘ | ₍k_{cat}/Kₘ₎^{NAD+} |
| SceFDH* | ND^{‡} | ND^{‡} | ND^{‡} | 36 ± 5 | 6.5 ± 0.4 | 0.18 | <3.3 × 10⁻¹⁰ |
| PseFDH* | >0.4M | ND^{‡} | ND^{‡} | 60 ± 5 | 10.0 ± 0.6 | 0.17 | 4.2 × 10⁻⁴ |
| | | | | | | | (0.3M formate) |
| CboFDH** | >38 | 4 × 10⁻⁵ | <1.05 × 10⁻⁶ | 0.015 ± 0.01 | 3.7 ± 0.1 | 246.7 | 4.26 × 10⁻⁹ |

TABLE 2
Kinetic Parameters for mutant FDHs

NAD⁺

| | | NADP⁺ | | | | | | (k_{cat}/Kₘ₎^{NADP+}/ |
|---|---|---|---|---|---|---|---|---|
| Mutants | | Kₘ (mM) | k_{cat} (min⁻¹) | k_{cat}/Kₘ | Kₘ (mM) | k_{cat} (min⁻¹) | k_{cat}/Kₘ | ₍k_{cat}/Kₘ₎^{NAD+} |
| D195Q CmetFDH | | 3.8 ± 0.6 | 83 ± 6 | 21.84 | 1.3 ± 0.1 | 100 ± 2 | 76.92 | 0.28 |
| | | | | | | | | (0.18M formate) |
| Q223D Bsp184FDH | | 111 ± 30 | 26 ± 6 | 0.23 | 0.2 ± 0.0 | 109 ± 1 | 545 | 4.22 × 10⁻⁴ |
| | | | | | | | | (0.18M formate) |
| Q223D Bsp383FDH | | 32 ± 8 | 11 ± 2 | 0.34 | 0.2 ± 0.0 | 114 ± 1 | 570 | 5.96 × 10⁻⁴ |
| | | | | | | | | (0.18M formate) |
| | | | | | | | | (k_{cat}/Kₘ₎^{NADP+}/ |
| | | Kₘ (μM) | k_{cat} (s⁻¹) | k_{cat}/Kₘ | Kₘ (μM) | k_{cat} (s⁻¹) | k_{cat}/Kₘ | ₍k_{cat}/Kₘ₎^{NAD+} |
| D195S CmetFDH* | | ND^{‡} | ND^{‡} | ND^{‡} | 4700 ± 300 | 1.6 ± 0.1 | 3.4 × 10⁻⁴ | 0.024 |
| | | | | | | | | (0.2M formate) |
| D197A/Y198R | 0.25M | 4500 ± 500 | 0.13 ± 0.01 | 2.9 × 10⁻⁵ | 7600 ± 800 | 0.095 ± 0.01 | 1.2 × 10⁻⁵ | 2.4 |
| SceFDH* | formate | | | | | | | |
| | 0.5M | 7600 ± 900 | 0.16 ± 0.02 | 2.1 × 10⁻⁵ | 8400 ± 900 | 0.12 ± 0.02 | 1.4 × 10⁻⁵ | 1.5 |
| | formate | | | | | | | |

| Mutant NADP⁺-specefic | 150 ± 25 | 2.5 ± 0.15 | 0.017 | 1000 ± 150 | 5.0 ± 0.4 | 0.005 | 3.4 |
|---|---|---|---|---|---|---|---|
| PseFDH* | | | | | | | (0.3M formate) |
| | | | | | | | (k_{cat}/Kₘ₎^{NADP+}/ |
| | Kₘ (mM) | k_{cat} (s⁻¹) | k_{cat}/Kₘ | Kₘ (mM) | k_{cat} (s⁻¹) | k_{cat}/Kₘ | ₍k_{cat}/Kₘ₎^{NAD+} |
| D195S CboFDH** | 6.2 ± 0.1 | 0.34 ± 0.03 | 0.055 | 1.5 ± 0.05 | 0.34 ± 0.03 | 0.227 | 0.242 |
| D195N CboFDH** | 13.2 ± 0.3 | 0.26 ± 0.02 | 0.0196 | 5.01 ± 0.2 | 0.21 ± 0.02 | 0.0419 | 0.468 |
| D195A CboFDH** | 3.3 ± 0.2 | 0.052 ± 0.01 | 0.0157 | 4.8 ± 0.1 | 0.76 ± 0.04 | 0.158 | 0.099 |
| D195Q CboFDH** | 4.5 ± 0.2 | 0.26 ± 0.02 | 0.058 | 0.96 ± 0.06 | 0.26 ± 0.02 | 0.271 | 0.214 |
| D195Q/T196S CboFDH** | 6.2 ± 0.3 | 0.34 ± 0.02 | 0.055 | 5.1 ± 0.06 | 0.4 ± 0.03 | 0.078 | 0.705 |
| D195Q/Y196P CboFDH** | 3.7 ± 0.2 | 0.34 ± 0.03 | 0.092 | 0.13 ± 0.01 | 0.87 ± 0.04 | 6.69 | 0.0138 |
| D915Q/Y196H CboFDH** | 1.7 ± 0.08 | 0.44 ± 0.03 | 0.26 | 1.8 ± 0.09 | 0.49 ± 0.03 | 0.27 | 0.96 |

*ref: data obtained from Serov et al. (2002)
**ref: data obtained from Andreadeli et al. (2008)
^{†}NR, not reported
^{‡}ND, not detectable

TABLE 3
Steady-State Kinetics Parameters of the FDHs Catalyzed Formate Oxidation
Reactions with NAD⁺ and NADP⁺

| | NADP⁺ | | | NAD⁺ | | |
|---|---|---|---|---|---|---|
| Wild Types | K_{M} (mM) | k_{cat} (min⁻¹) | k_{cat}/K_{M}^{formate} | K_{M} (mM) | k_{cat} (min⁻¹) | k_{cat}/K_{M}^{formate} |
| CmetFDH | — | — | — | 8.29 ± 1.00 | 419.25 ± 17.57 | 50.57 ± 6.06 |
| Bsp184FDH | 156.98 ± 30.00 | 132.44 ± 15.32 | 0.84 | 99.18 ± 7.11 | 20.40 ± 0.74 | 0.21 |
| Bsp383FDH | 126.79 ± 16.61 | 366.89 ± 26.84 | 2.89 | 50.13 ± 2.69 | 46.32 ± 0.95 | 0.92 |

TABLE 4
Kinetic Parameters for Previous Cofactor Engineering Studies

| | | | | NAD⁺ | | | |
|---|---|---|---|---|---|---|---|
| | NADP⁺ | | | | | | (k_{cat}/Kₘ₎^{NADP+}/ |
| ADHs | Kₘ (mM) | k_{cat} (min⁻¹) | k_{cat}/Kₘ | Kₘ (mM) | k_{cat} (min⁻¹) | k_{cat}/Kₘ | ₍k_{cat}/Kₘ₎^{NAD+} |
| WT_KlADH^{III}[1] | 2.64 | 1160 | 440 | 0.39 | 24500 | 62800 | 0.007 |
| G226A KlADH^{III}[1] | 2.77 | 1640 | 592 | 1.00 | 24300 | 24300 | 0.024 |
| A275F KlADH^{III}[1] | 6.21 | 659 | 106 | 0.98 | 17100 | 17400 | 0.006 |
| G226A/A275F KlADH^{III}[1] | 5.50 | 1540 | 280 | 0.57 | 8430 | 14800 | 0.019 |
| | Kₘ (mM) | k_{cat} (s⁻¹) | k_{cat}/Kₘ | Kₘ (mM) | k_{cat} (s⁻¹) | k_{cat}/Kₘ | |
| WT_DroADH[2] | 3.09 ± 0.70 | 0.715 | 0.230 | 0.048 ± 0.001 | 10.2 | 213 | 1.08 × 10⁻³ |
| A46R DroADH[2] | 1.27 ± 0.10 | 1.9 | 1.5 | 0.057 ± 0.003 | 7.3 | 127 | 0.012 |
| D39N DroADH[2] | 0.050 ± 0.003 | 6.8 | 136 | 0.073 ± 0.002 | 15.3 | 210 | 0.65 |
| D39N/A46R DroADH[2] | 0.100 ± 0.003 | 5.7 | 58 | 0.260 ± 0.003 | 15.0 | 58 | 1 |
| WT_SceADH[9] | NR^{‡} | NR^{‡} | NR^{‡} | 0.16 | 360 | 2300 | — |
| D223G SceADH[9] | 20 | 54 | 2.7 | 18 | 38 | 2.1 | 1.29 |
| | | | | | | | (k_{cat}/Kₘ₎^{NADP+}/ |
| AlaDHs | Kₘ (mM) | k_{cat} (s⁻¹) | k_{cat}/Kₘ | Kₘ (mM) | k_{cat} (s⁻¹) | k_{cat}/Kₘ | ₍k_{cat}/Kₘ₎^{NAD+} |
| WT_SheAlaDH[3] | 4.00 | 1.60 | 0.4 | 0.035 | 34.7 | 991 | 4.03 × 10⁻⁴ |
| R199I SheAlaDH[3] | ND^{†} | 0.00 | 0.00 | 0.01 | 9.50 | 950 | 0 |
| D198G SheAlaDH[3] | 0.67 | 24.1 | 36.0 | 3.0 | 41. | 13.7 | 2.63 |
| D198A SheAlaDH[3] | 0.35 | 37.8 | 108 | 5.6 | 43.5 | 7.8 | 13.85 |
| D198V SheAlaDH[3] | 1.1 | 50.2 | 45.6 | 5.2 | 41.2 | 7.9 | 5.77 |
| D198L SheAlaDH[3] | 2.1 | 4.8 | 2.3 | 11.4 | 1.9 | 0.2 | 11.5 |
| WT_PlaAlaDH[3] | ND^{†} | 0.00 | 0.00 | 0.036 | 95.5 | 2650 | 0 |
| I198R PlaAlaDH[3] | 2.8 | 1.3 | 0.46 | 0.15 | 95.4 | 640 | 7.19 × 10⁻⁴ |
| | | | | | | | (k_{cat}/Kₘ₎^{NADP+}/ |
| LDHs | Kₘ (μM) | k_{cat} (s⁻¹) | k_{cat}/Kₘ | Kₘ (μM) | k_{cat} (s⁻¹) | k_{cat}/Kₘ | ₍k_{cat}/Kₘ₎^{NAD+} |
| WT_BsLDH[4] | NR^{‡} | NR^{‡} | NR^{‡} | 105 ± 8.8 | 3 ± 0.80 | 0.30 | — |
| (with FBP as activator) | | | | | | | |
| I37K/D38S BsLDH[4] | 4700 ± 630 | 23 ± 1.6 | 0.005 | 8.1 ± 0.52 | 23 ± 0.26 | 2.8 | 1.79 × 10⁻³ |
| (with FBP as activator) | | | | | | | |
| | | | | NAD⁺ | | | |
| | NADP⁺ | | | | | | (k_{cat}/Kₘ₎^{NADP+}/ |
| G6PDHs | Kₘ (mM) | k_{cat} (min⁻¹) | k_{cat}/Kₘ | Kₘ (mM) | k_{cat} (min⁻¹) | k_{cat}/Kₘ | ₍k_{cat}/Kₘ₎^{NAD+} |
| WT_AhaG6PDH[5] | 0.104 ± 0.0091 | 1.28 × 10⁵ ± 3500 | 1.23 × 10⁶ | 0.340 ± 0.053 | 7.7 × 10⁴ ± 6500 | 2.27 × 10⁵ | 5.41 |
| | | | | | | | (k_{cat}/Kₘ₎^{NADP+}/ |
| 17β-HSDHs | Kₘ (μM) | k_{cat} (s⁻¹) | k_{cat}/Kₘ | Kₘ (μM) | k_{cat} (s⁻¹) | k_{cat}/Kₘ | ₍k_{cat}/Kₘ₎^{NAD+} |
| WT_17β-HSDHs[6] | 0.6 ± 0.01 | 4.2 ± 0.2 | 7.0 | 11.8 ± 0.8 | 7.5 ± 0.1 | 0.6 | 11.7 |
| S12K 17β-HSDHs[6] | 0.4 ± 0.08 | 2.5 ± 0.06 | 6.0 | 96 ± 0.2 | 2.6 ± 0.2 | 0.03 | 200.0 |
| L36D 17β-HSDHs[6] | 1475 ± 35 | 16.7 ± 2.2 | 0.01 | 83.0 ± 5.0 | 13.9 ± 0.8 | 0.2 | 0.05 |
| | | | | | | | (k_{cat}/Kₘ₎^{NADP+}/ |
| XDHs | Kₘ (mM) | k_{cat} (min⁻¹) | k_{cat}/Kₘ | Kₘ (mM) | k_{cat} (min⁻¹) | k_{cat}/Kₘ | ₍k_{cat}/Kₘ₎^{NAD+} |
| WT_PstXDH[7] | 170 ± 16 | 110 ± 10 | 0.65 | 0.381 ± 0.030 | 1050 ± 30 | 2760 | 2.36 × 10⁻⁴ |
| ARS_PstXDH[7] | 0.897 ± 0.036 | 2500 ± 50 | 2790 | 1.30 ± 0.13 | 240 ± 17 | 181 | 15.41 |
| ART_PstXDH[7] | 0.638 ± 0.031 | 1970 ± 50 | 3090 | 0.265 ± 0.016 | 310 ± 13 | 1170 | 2.64 |
| ARSdR_PstXDH[7] | 1.38 ± 0.11 | 3840 ± 270 | 2790 | 17.3 ± 2.1 | 1430 ± 160 | 84 | 33.2 |
| C4/ARS_PstXDH[7] | 1.18 ± 0.06 | 12600 ± 400 | 10700 | 23.5 ± 1.4 | 1770 ± 100 | 75.3 | 142.1 |
| C4/ARSdR_PstXDH[7] | 1.04 ± 0.04 | 11000 ± 400 | 10500 | 7.60 ± 0.5 | 790 ± 40 | 104 | 100.96 |
| | | | | | | | (k_{cat}/Kₘ₎^{NADP+}/ |
| PTDH | Kₘ (μM) | k_{cat} (min⁻¹) | k_{cat}/Kₘ | Kₘ (μM) | k_{cat} (min⁻¹) | k_{cat}/Kₘ | ₍k_{cat}/Kₘ₎^{NAD+} |
| WT_PstPTDH[8] | 2510 ± 410 | 84.6 ± 0.5 | 0.0337 | 53 ± 9.0 | 175.8 ± 8.4 | 3.3 | 0.0102 |
| E175A PstPTDH[8] | 144 ± 14 | 130.8 ± 0.4 | 0.91 | 16 ± 0.8 | 210.0 ± 0.3 | 13.1 | 0.07 |
| A176R PstPTDH[8] | 77 ± 8.4 | 130.8 ± 0.4 | 1.7 | 60 ± 7.0 | 256.8 ± 0.5 | 4.3 | 0.40 |
| E175A/A176R | 3.5 ± 0.5 | 114.0 ± 0.5 | 32.6 | 20 ± 1.3 | 236.4 ± 0.5 | 11.8 | 2.76 |
| PstPTDH[8] | | | | | | | |
| | | | | | | | (k_{cat}/Kₘ₎^{NADP+}/ |
| GAPDH | Kₘ (mM) | k_{cat} (s⁻¹) | k_{cat}/Kₘ | Kₘ (mM) | k_{cat} (s⁻¹) | k_{cat}/Kₘ | ₍k_{cat}/Kₘ₎^{NAD+} |
| WT_BstGAPDH[9] | NR^{‡} | NR^{‡} | NR^{‡} | 0.15 | 280 | 1900 | — |
| L187A/P188S | 7.1 | 58 | 8.2 | 0.35 | 280 | 800 | 0.01 |
| BstGADPH[9] | | | | | | | |

Data obtained from [1] Brisdelli et al. (2004); [2] Chen et al. (1994) & (1991) [3] Ashida et al. (2004); [4] Flores et al. (2005); [5] Ragunathan et al. (1994); [6] Huang et al. (2001); [7] Watanabe et al. (2005) [mutant name referred as ref]; [8] Woodyer et al. (2003); [9] Fan et al.(1991)
Abbreviations:
ADHs, alcohol dehydrogenases;
KIADH^{III}, ADH III from Kluyveromyces lactis;
DroADH, ADH from Drosophila;
SceADH, ADH from Saccharomyces cerevisiae;
AlaDH, alanine dehydrogenases;
SheAlaDH, AlaDH from Shewanella sp. Ac10;
PlaAlaDH, AlaDH from Phromidium lapideum;
LDHs, lactate dehydrogenase;
BsLDH, D-lactate dehydrogenase from Bacillus stearothermophilus;
FBP, D-fructose 1,6-diphosphate;
AhaG6PDH, gucose-6-phosphate dehydrogenases from Acetobacter hansenii;
17β-HSDHs, human estrogenic 17β-hydroxysteroid dehydrogenases;
PstXDH, xylitol dehydrogenases from Pichia stipitis;
PstPTDH, phosphate dehydrogenase from Pseudomonas stutzeri;
BstGADPH, glyceraldehydes-3-phosphate dehydrogenase from Bacillus stearothermophilus;
WT, wild type.
^{†}ND, not detectable
^{‡}NR, not reported

TABLE 5
Comparison of Kinetic Properties of Recombinant FDHs and their Mutants.
A unit is defined as μmol of substrate (formate) oxidized per min in the presence of co-factor

| | | | | | | Coenzyme Preference | |
|---|---|---|---|---|---|---|---|
| Enzyme | Mutations | NAD⁺ | | NADP⁺ | | (kcat/Km) ^{NAD+}/(kcat/Km) ^{NADP+} | Reference |
| Pseudomonas sp. 101 | Wt, PseFDH | 6-8 | U/mg | | | 2400 | Tiskov, et al, |
| | T5M8 (patented) | — | | 1.3-1.6 | U/mg | | Biochemistry(2004), |
| | T5M9 (patented) | — | | 2.5 | U/mg | | 69, 1252-67 |
| | T5M9-10 (patented) | — | | 2.5 | U/mg | | |
| C. bodinii | Wt, CboFDH | 2.2 | U/mg | 0.0013 | U/mg | | Tiskov, et al, |
| | D195S | 1.5 | U/mg | 0.083 | U/mg | | Biochemistry(2004), |
| | D195S/Y196H | 1.3 | U/mg | 0.19 | U/mg | | 69, 1252-67 |
| | D195S/Y196H/K356T | 1.3 | U/mg | 0.36 | U/mg | | |
| C. methylica | wt | (k_{cat} = 1.6 s⁻¹) | | ND | | 250,000 | Biotech Lett, |
| | D195S | | | | | 8.3 | 23, 283-87 |
| S. cerevisiae | wt | | | | | 0.30 × 10¹⁰ | |
| Bsp 184 FDH | Wt, Bsp184FDH | 0.48 | U/mg | 3.12 | U/mg | 0.23 | This work |
| Bsp383 FDH | Wt, Bsp383FDH | 1.09 | U/mg | 8.64 | U/mg | 0.29 | This work |

The amino acid sequence alignment of FDHs in FIG. 18 demonstrates that the aspartate that binds the hydroxyl group of the adenine ribose moiety of NAD⁺ is highly conserved in many NADH-dependent dehydrogenases, but not in the novel NADPH-dependent FDHs described here.

To gain a further insight into the molecular origin of the cofactor specificity of FDH enzymes, the 3D structure of BspFDH2 was determined in native, binary (NADP⁺) and ternary (NADP⁺/formate/azide) forms using X-ray crystallography at resolutions between 1.5 and 2.5 Å. At a global structural level, the topology of BspFDH2 is similar to that observed for previous NAD⁺-specific FDHs (Lamzin, V. S. et al., Journal of Molecular Biology 236, 759-785 (1994)). The structure is dimeric, each monomer composed of a two-domain structure in which NADP⁺ binding occurs predominantly on the C-terminal nucleotide binding fold with the catalytic centre at the domain interface. FIG. 3 shows the structural basis for co-factor recognition, and manipulation, in Burkholderia sp. 383 FDH BspFDH2). FIG. 3 (a) shows the FDH dimer with the NADP/formate ligand shown in ball and stick with electron density.

In the case of BspFDH2, the nicotinamide ring is disordered in the binary complex and is only observed ordered (see FIG. 1b) in the ternary complex. Crystals grown under high formate concentration (˜500 mM formate, 0.5 mM azide) revealed clear density for the reductant ligand (whose identity cannot be formally resolved at this resolution). The central atom lies 2.7 Å from C4 of the nicotinamide ring, perfectly poised for hydride abstraction.

When formate is introduced into the BspFDH2 structure, the recognition elements that interact directly with the two carboxylate oxygens with O1 making H-bonds to the main chain amide of Ile124 and to the side-chain amide of Asn148, can be immediately seen. Whilst the majority of the interactions of BspFDH2 are as observed for the NAD⁺-specific FDHs, significant and key differences are revealed in the adenine-ribose recognition loop, which in BspFDH2 is selective for the phosphoribose of NADP⁺. FIG. 3(b) shows the phosphate recognition in the adenine-ribose recognition loop revealed through the overlap of Bsp383FDH (with electron density for part of the NADP⁺ moiety shown) with an obligate NAD⁺ utilising FDH (Pseudomonas sp. 101; PDB code 2NAD).

In NAD⁺-specific FDHs, ribose recognition is conferred, predominantly, by an aspartate (at a position corresponding to position 223 in the Bsp enzymes) which makes H-bonds to both ribose O2 and O3 hydroxyl groups. In the BspFDH2, NADP⁺-selective enzyme, glutamine—a larger amino acid residue—is found in the corresponding position (Gln223) which, counter-intuitively, provides recognition of the phosphoriboside (see FIG. 3(b)). The side-chain of glutamine, instead of interacting with the ribose, provides a “super-secondary structural” hydrogen-bonding network from the main-chain amide of Ala201, to the side-chain carbonyl of Gln223, with the amide of this side-chain hydrogen-bonding to the main-chain carbonyl of His225. The bulge thus inserted into this loop, changes the environment of Arg224 in BspFDH2 which is now able to interact, via NH₂ and N-protonated guanidino nitrogens, with two of the phosphate oxygens of the phosphoribose (see FIG. 3b). The remaining phosphate oxygen also accepts a hydrogen-bond from the main chain amide of His225.

While specificity for phosphate harnesses an arginine (Arg224 in BspFDH2) present in both NAD⁺ and NADP⁺ dependent enzymes, it appears that the Asp-to-Gln (at a position corresponding to amino acid 223 in BspFHD2) change allows Arg224 to make the phosphate-recognizing interactions and to accommodate subtle conformational changes in the riboside ring. Thus, Gln 223 may be a key recognition element for the phosphate of NADP⁺, with Arg 224 becoming more ordered and interacting with the phosphate.

Amino acid sequence alignment of several FDHs (FIG. 18) reveals that the aspartate that binds the hydroxyl group of the adenine ribose moiety of NAD⁺ is highly conserved in many NADH-dependent dehydrogenases, but not so in the NADPH-dependent FDHs.

The importance of this key determinant of the molecular basis for cofactor preferences was confirmed by the re-engineering of several FDHs from both specificity classes.

The re-engineering involved the mutation of the amino acid in other FDH enzymes that corresponds to the amino acid at position 223 in BspFDH2. More specifically, the amino acid residue at position 195 in the NADH-dependent wild-type (wt) Candida methylica FDH enzyme, CmetFDH-wt, was mutated to create the mutant enzyme CmetFDH-D195Q.

‘Reverse’ Gln to Asp mutations were created at the corresponding residue (position 223) in both BcenFDH1 and BspFDH2. Although CmetFDH-D195Q still favoured NAD⁺, the (kcat/Km)NADP⁺/(kcat/Km)NAD⁺ ratio was shifted almost 5000-fold through this single change (See FIG. 2).

Even more surprisingly, mutation of the glutamine at position 223 to aspartic acid in both BcenFDH1 and BspFDH2 dramatically changed the cofactor preference from NADP⁺ to NAD⁺ (see FIG. 2). FIG. 3(c) shows manipulation of co-factor specificity through the Gln223-Asp223 mutation of the BspFDH2. Introduction of the aspartate inverts the co-factor specificity, such that kcat/Km (NAD+)/kcat/Km (NADP⁺) is >6000. The 3D structure of the BspFDH2 Q>D variant, BspFDH2-Q223D, this time in binary complex with NAD⁺ (FIG. 3c) confirms that the mutation reverts the ribose loop back to that observed in NAD⁺-specific enzymes, in which the aspartate interacts with O2 and O3 of the ribose with the loop conformation leaving Arg223 disordered in the solvent.

Thus, NADH vs NADPH cofactor control is both steric (counter-intuitively, a larger Gln residue perturbs cofactor and active site geometries to accommodate the larger NADPH O-3′ phosphate substituent) and electrostatic (by removing the negative charge of the Asp carboxylate COO— side chain a negatively charged O-3′ phosphate is no longer subject to coulombic repulsion).

The results presented here demonstrate that a 10⁵-fold cofactor preference switch, both to and from NADPH, can be achieved in several FDHs by changing only one or a few residues.

The directed change in coenzyme specificity of a selected FDH has great importance for commercial biocatalysis, because until now the use of FDHs for efficient regeneration of the NADPH cofactor has been not possible. However, the reactions catalysed by BcenFDH1 and BspFDH2 fit the near ideal requirements for the NAD(P)H regeneration.

Since the reaction of formate oxidation yields CO₂, which can be easily removed from the system, this not only allows thermodynamically-coupled reactions to be forced to obtain desired products with 99-100% yield, but also simplifies downstream processing, resulting in dramatically lowered production costs. Formate salt is also cheap and readily available and does not inhibit BcenFDH1 or BspFDH2.

Furthermore, BcenFDH1 and BspFDH2 activities change little in the pH range 4.5-9 (FIGS. 13A and 13B) and the enzymes are still highly active even at pHs as low 4 or as high as 10.5. Thus, BcenFDH1 and BspFDH2 can be used effectively in combination with a large number of enzymes that show activity in this broad range. BcenFDH1 and BspFDH2 are also highly stable enzymes (both are stable at room temperature for weeks and no activity was lost upon incubation at 55° C. for 2 hours (although both denature at 80° C.) and can be used for long periods of time. FIGS. 19 and 20 illustrate the thermostability of BcenFDH1 and BspFDH2.

Using the recombinant system described here with its high level of protein expression (˜20 mg/L), and easy purification, BcenFDH1 and BspFDH2 can also be prepared in a very cost effective manner.

Without being bound to any particular theory, from these results it would appear that Gln223 or the amino acid at the position corresponding to Gln223 in BspFDH2 plays a critical roles in NADP⁺/NAD⁺ specificity.

### Examples of the Use of BspFDH Enzymes

To demonstrate the synthetic utility of novel BspFDH enzymes, various important NADPH-dependent oxidoreductases were coupled with BspFDH2 to obtain highly valued chiral intermediates and fine chemicals (see FIG. 4 and the Examples below). The four strikingly varied redox transformations that are difficult to achieve by conventional methods in organic chemistry, successfully demonstrated the power of BspFDH-coupled biocatalysis; notably, the wide pH tolerance of BspFDH2 allowed reactions at quite different pH optima (pH 7.5 (CHMO); 6.6 (βKR); 9.0 (DAADH)). In all cases, control experiments in which BspFDH was omitted yielded only un-reacted starting material without any product.

### EXAMPLE 1

### Cytochrome P450 BM3/BspFDH2 Coupled Oxidation

The cytochrome P450 system is a ubiquitous superfamily of monooxygenases that is present in plants, animals, and prokaryotes. The human genome encodes more than 50 members of the family, whereas the genome of the plant Arabidopsis encodes more than 250 members.

In mammals, the cytochrome P450 system is located mainly in the endoplasmic reticulum of the liver and small intestine and plays an important role in the detoxification of foreign substances (xenobiotic compounds) by oxidative metabolism. The enzymes catalyzing these reactions are called monooxygenases (or mixed-function oxygenases). The resulting hydroxylated products have various potential applications as polymer building blocks, as intermediates in antibiotic synthesis and perfume ingredients.

The following schema gives an overview of reactions catalysed by P450-monooxygenases (adapted from Bornscheuer et al {Bornscheuer, 2005, Eng. Life Sci, 5, 309}) in which novel NADP⁺ specific FDH enzymes may be useful.

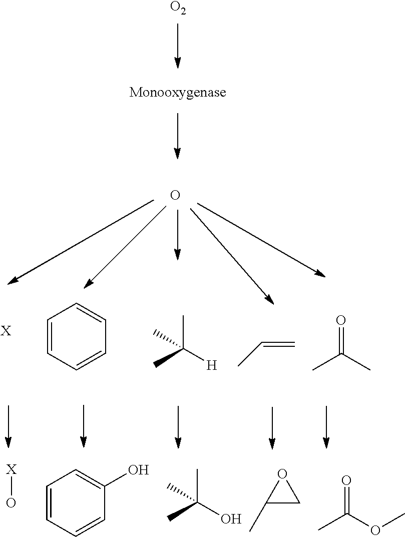

To date, the strict requirement for the cofactor NADPH rather than NADH has limited the economic use of these potentially important enzymes (in 2008 the price of NADPH was £2700/mmol).

### EXAMPLE 1a

### Wild Type Cytochrome P450 BM3/BspFDH2 Coupled Oxidation of 1-propylbenzene (1a)

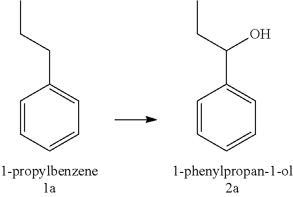

To demonstrate the commercial use of BspFDH2, the regio- and stereo-selective insertion of an oxygen atom into propylbenzene (1) to obtain 1-phenyl-1-propanol (2) was performed (>99% conversion, >90% e.e, complete within 2 hours) by coupling the enzymatic action of BspFDH2 with that of cytochrome-P450-monooxygenase BM-3. (Li, Q.-S. et al., FEBS Letters 508, 249-252 (2001)) In this oxygen C—H reaction (FIG. 4a), one oxygen atom from an atmospheric O₂ molecule is reduced to water by NADPH leaving the other ready for delivery to substrate. All P450s are NADPH-dependent. NADP⁺ thus formed is then regenerated to NADPH by the action of BspFDH2 using formate. The overall atomic transformation is therefore the splitting of O₂ with a hydride ion from formate using NADP⁺ as a hydride shuttle.

To demonstrate NADPH regeneration following oxidation of 1-propylbenzene the following experiment was performed. 200 μL of 1-propylbenzene (1a above, 1M in DMSO), sodium formate (3 M, 2 mL) and bovine serum albumin (100 mg/mL, 1 mL) were added in potassium phosphate buffer (100 mM, pH 7.6, 2 mL) and the reaction mixture was shaken for 3 min. NADP+ (10 mM, 1 mL) and BspFDH2 (17 mg/mL, 500 μL) were added and the reaction shaken for 30 minutes at room temperature. After 30 minutes, wild type cytochrome P450-BM3 (WT P450 BM3, 2.4 mg/mL, 300 μL) was added and the reaction shaken at room temperature. The reaction was monitored by GC-MS (Rt˜3.2 minutes). After 15 hr, the reaction mixture was extracted by DCM (3×1 mL) and the combined organic phase was dried and the solvent removed to afford 1-phenylpropan-1-ol (2a above, >99% conversion based on GC chromatogram). The GC-MS data were collected in full-scan mode (m/z 50-300) using a Zebron column (Phase ZB-5, 0.25 mm×15 m, 0.25 μm film thickness; Phenomenex) on a GCT system (GC: Agilent G890 series, MS: Micromass). The following GC program was used: 80° C. (2 min hold), 80-280° C. (10° C./min) and 280° C. (5 min hold). Chiral-phase GC was performed on a cyclodextrin column (0.25 mm×30 m, thickness 0.25 μm), using helium as a carrier gas (flow=1.2 mL/min, injector T=220° C.) and flame ionization detection (FID, T=250° C.) and programmed to an initial temperature of 40° C. and then ramped 10° C. per minute until 80° C. After holding at 80° C. for 5 minutes, the program was ramped 1° C. per minute until 140° C. IR, vₘₐₓ (thin film), 3600-3100 (br, O—H), 1493 (m), 1453 (m), {1095 and 1013 (m) C—O}, 700 (s, monosubst.) cm⁻¹. ¹H-NMR (400 MHz, Chloroform-d): δ ppm 0.90 (t, J=7.45 Hz, 3H), 1.66-1.87 (m, 2H), 4.53 (t, J=6.57, 2.78 Hz, 1H) 7.30-7.37 (m, 4H). ¹³C-NMR (400 MHz, Chloroform-d): δ ppm 10.0, 31.7, 75.8, 125.9, 127.3, 128.2, 144.5. GC-MS (Cr): m/z Calcd for C₉H₁₂O [M]⁺=136, C₉H₁₃O [M+H]⁺=137. Found: 136 (M)⁺, 137 (M+H)⁺. Chiral-phase GC-FID: (tR=29.07 minutes, tS=30.17 minutes), >99% e.e. of S. FIG. 16 shows chiral gas chromatograms of 1-propylbenzene oxidation by WT P450 BM3.

### EXAMPLE 1b

### Cytochrome P450 BM3/BspFDH2 Coupled Oxidation of Octane (1b)

To further demonstrate the commercial use of BspFDH2, NADPH regeneration following the oxidation of octane was studied.

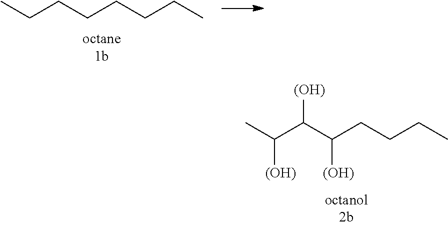

5 μL of octane (1b, 100 mM in DMSO), sodium formate (3 M, 50 μL) and bovine serum albumin (1 μL, 100 mg/mL) were added in Tris-HCl buffer (50 mM, pH 7.4, 774 μL, final volume: 1 mL) and the reaction mixture was shaken for 3 min. NADP⁺ (100 mM, 100 μL) and BspFDH2 (1 mg/mL, 50 μL) were then added and the reaction shaken for 30 minutes at room temperature. After 30 minutes, wild type cytochrome P450 Bm3 (WT P450 BM3, 2.4 mg/mL, 20 μL) was added and the reaction shaken at room temperature. After 4 hr, the reaction mixture was extracted by DCM (2×400 μL) and the combined organic phase was dried and the solvent removed to afford octanol mixtures (2b). GC was performed on a cyclodextrin column (0.25 mm×30 m, thickness 0.25 μm), using helium as a carrier gas (flow=1.2 mL/min, injector T=220° C.) and flame ionization detection (FID, T=250° C.) and programmed to an initial temperature of 40° C. and then ramped 10° C. per minute until 80° C. After holding at 80° C. for 1 minute, the program was ramped 5° C. per minute until 140° C. The peaks of octanol mixtures were assigned by standards obtained from Sigma. Use of cytochrome P450-BM3 mutant A330P4/BspFDH2 coupled oxidation of octane followed the same conditions as above to obtain octanol mixtures. Gas chromatograms of octane oxidation by wild type and mutant P450 BM3 are shown in FIG. 17.

### EXAMPLE 2

### Oxygen C—C Bond Insertion

### Such as Cyclohexanone Monoxygenase (CHMO)/BspFDH2 Coupled Baeyer-Villiger Oxidation of Cyclohexanone (3)

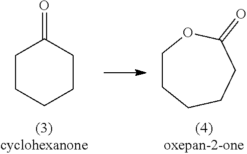 [SMILES: O=C1CCCCC1 ; O=C1CCCCCO1]

Several organisms have been identified which catalyse the above reaction of cyclohexanone (3) to oxepan-2-one. Many flavin dependent monooxyhenases have been reported to accept a multitude of non-natural substrates. Cyclohexanone monooxygenase from Acinetobacter NCIB 9871 is one of the most widely studied asymmetric Baeyer-Viliger enzymes (Taschner M J., et al., J. Am. Chem. Soc, 1988, 110, 6892). Various ketones have been examined, and in many cases yields and enantioselectivity for this type of conversion are extremely high.

Some representative CHMO substrates are as follows:

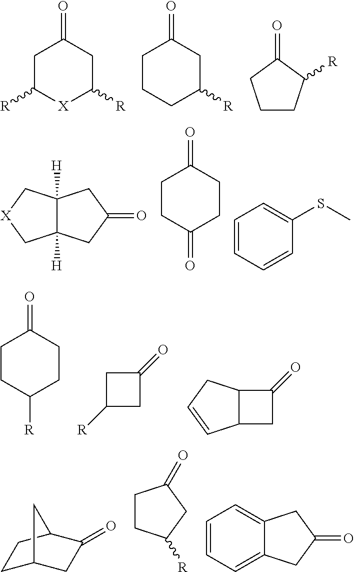

By coupling the enzymatic action of BspFDH2 with that of cyclohexanone monooxygenase (CHMO), oxygen C—C bond insertion, a Baeyer-Viliger (B-V) oxidation (see FIG. 4b), converted (>99% conversion, complete within 4 hours) cyclohexanone (3) to caprolactone (4) with NADPH regeneration.

To demonstrate NADPH regeneration following cyclohexanone oxidation 200 μL of cyclohexanone (3, 1M in DMSO), sodium formate (3 M, 2 mL) and bovine serum albumin (100 mg/mL, 1 mL) were added in potassium phosphate buffer (100 mM, pH 7.6, 2 mL) and the reaction mixture was shaken for 3 min. NADP+ (20 mM, 1 mL) and BspFDH2 (17 mg/mL, 500 μL) were then added and the reaction shaken for 30 minutes at room temperature. After 30 minutes, pre-dialysed CHMO (2 mg/mL, 300 μL) was then added and the reaction shaken at 37° C. The reaction was monitored by GC-MS (Rt˜3.1 minutes). After 4 hr, the reaction mixture was extracted by DCM (3×1 mL) and the combined organic phase was dried and the solvent removed to afford oxepan-2-one (4 above, >99% conversion based on GC chromatogram).

The GC-MS data were collected as example 1a. IR,vₘₐₓ (thin film), 1730 (C═O, m) cm⁻¹. ¹H-NMR (400 MHz, Chloroform-d): δ ppm 1.70-1.89 (m, 6H), 2.60-2.65 (m, 2H), 4.19-4.21 (m, 2H). ¹³C-NMR (400 MHz, Chloroform-d): δ ppm 22.85, 28.88, 29.20, 34.48, 69.24, 176.18. GC-MS (Cl⁺): m/z Calcd. for C₆H₁₀O₂ [M]⁺=114, C₆H₁₁O₂ [M+H]⁺=115, C₆H₁₄NO₂ [M+NH₄]⁺=132. Found: 115 (M+H)⁺, 132 (M+NH₄)⁺.

### EXAMPLE 3

### Ketoreductase (KRED101)/BspFDH2 Coupled Reactions

Ketoreductases are valuable biocatalysts for chemo, regio- and diastereo-selective reductions/oxidations enabling resolutions and the synthesis of chiral alcohols from ketones, ketoacids, and ketoesters allowing synthesis of key pharmaceuticals ranging from anti-depressants to adrenergic drugs.

The reduction of a number of aldehydes is also catalyzed by these enzymes. Additionally, ten known human aldo-ketoreductases can turnover a vast range of substrates, including drugs, carcinogens, and reactive aldehydes. Their broad substrate tolerance make these particularly attractive synthetic tools with potentially wide applicability however the current key obstacle in the industrialisation of ketoreductase-catalyzed reactions is their requirement for the cofactor NADPH. The following schema illustrates the broad potential substrate application of FDH-catalysed NADPH regeneration in coupled KRED-catalysed carbonyl reduction:

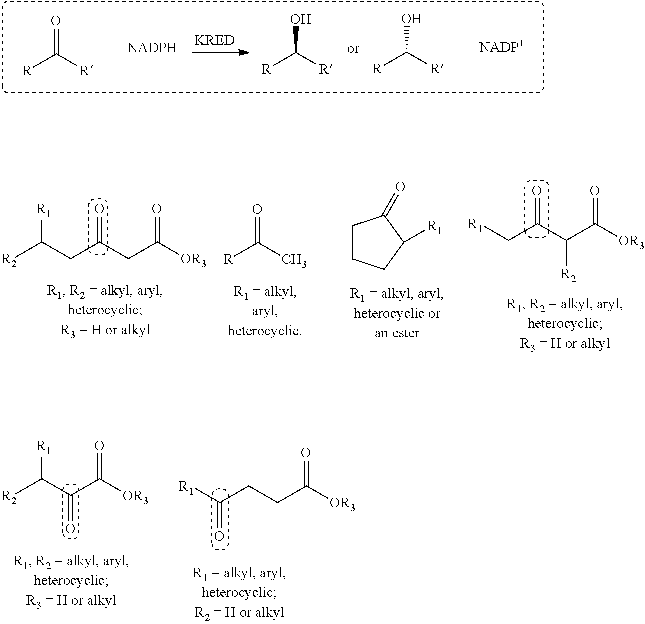

The results show that isolated ketoreductases can be screened against target ketones; the resulting discovered reactions can then be directly scaled up quickly to produce preparative amounts of chiral alcohols.

### Ketoreductase (KRED101)/BspFDH2 Coupled Reduction of Ethyl 4-chloro-3-oxobutanoate (5)

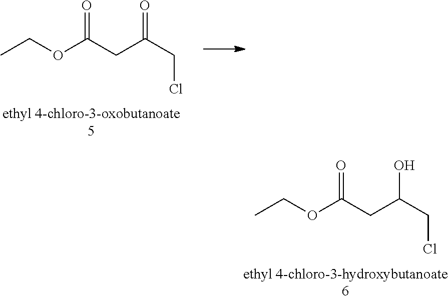

To demonstrate NADPH regeneration following reduction of ethyl 4-chloro-3-oxobutanoate 200 μL of ethyl 4-chloro-3-oxobutanoate (5 above, 1M in DMSO), sodium formate (3 M, 2 mL) and bovine serum albumin (100 mg/mL, 1 mL) were added in potassium phosphate buffer (100 mM, pH 6.6, 2 mL) and the reaction mixture was shaken for 3 min NADP (10 mM, 1 mL) and BspFDH2 (17 mg/mL, 500 μL) were then added and the reaction shaken for 30 minutes at room temperature. After 30 minutes, KRED101 (40 mg/mL, 300 μL) was added. The reaction was monitored by GC-MS (Rt˜3.1 minutes). After 15 hr, the reaction mixture was extracted by DCM (3×1 mL) and combined organic phases were dried and the solvent removed to afford ethyl 4-chloro-3-hydroxybutanoate (6 above, >99% conversion based on GC chromatogram and HPLC). The GC-MS data were collected as example 1a. Chiral HPLC was performed on a Chiralcel OB packed column (0.46 cm I.D.×25 cm L; Daicel Chemical Industries, Japan) at 217 nm, with an eluent of n-hexane/2-propanol (9.3:0.7, v/v) at a flow rate of 1 mL/min. IR,vₘₐₓ (thin film), 3600-3100 (br, O—H), 1731 (s, C═O) cm⁻¹. ¹H-NMR (400 MHz, Chloroform-d): δ ppm 1.29 (t, J=7.07 Hz, 3H), 2.55-2.70 (m, 2H), 3.62 (dd, J=5.31, 2.78 Hz, 2H) 4.19 (q, J=7.07 Hz, 2H), 4.23-4.30 (m, 1H). ¹³C-NMR (400 MHz, Chloroform-d): δ ppm 14.11, 38.41, 48.11, 61.01, 67.92, 171.78. GC-MS (Cl⁺): m/z Calcd for C₆H₁₂O₃Cl [M+H]⁺=167, C₆H₁₅O₃NCl [M+NH₄]⁺=184. Found: 167 (M+H)⁺, 184 (M+NH₄)⁺. HPLC-UV/Vis: (tR=7.9 minutes, tS=9.4 minutes), >99% e.e. of S.

As can be seen from the above example, stereoselective hydride delivery to ethyl 4-chloro-3-oxobutonate yielded [>99% conversion, >99% e.e. complete within 30 minutes] optically active D-(S)-4-chloro-3-hydroxybutanoate ethyl ester (6 above). D-(S)-4-chloro-3-hydroxybutanoate ethyl ester is a key chiral pharmaceutical intermediate used in the enantioselective synthesis of, for example, slagenin B and C and HMG-CoA reductase inhibitors. This may be accomplished by coupling the enzymatic action of BspFDH, or any other NADP⁺-specific FDH according to the invention, with that of beta-ketoreductase (βKR) KRED101. βKR-mediated chemo, regio- and diastereo-selective reductions of ketones, ketoacids, and ketoesters to chiral alcohols allows the synthesis of key pharmaceuticals ranging from anti-depressants to adrenergic drugs.

The oxidoreductase process may involve ketoreductase-mediated synthesis, such as for an unnatural amino acid synthesis (a variant of the Degussa tert-leucine synthesis. In particular, stereoselective reductive amination of 2-oxooctanic acid to D-hexylglycine may be achieved by coupling the enzymatic action of BspFDH, or any other NADP⁺-specific FDH according to the invention, with that of D-amino acid dehydrogenase (DAADH). D-amino acids have been used as key components in many biologically important compounds including antibiotics, fertility drugs, anticoagulants and pesticides. Ampicillin (containing D-phenylglycine) is currently produced on a scale of >5000 tons per year.

### EXAMPLE 4

### D-aminoacid Dehydrogenase (DAADH))/BspFDH1 Coupled Reactions

This enzyme system catalyzes the reductive amination of 2-ketoacids to 2-amino acids in the presence of an ammonia source in a manner that is often highly selective towards production of the D-enantiomer. This process therefore has a strong potential utility in D-amino acid synthesis but has a strict requirement for nicotinamide cofactor NADPH. The following represents D-amino acid synthesis using DAADH and NADPH:

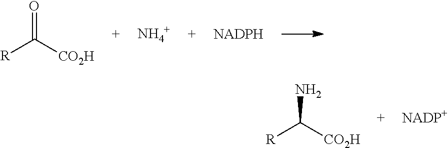

D-amino acids are found in nature, in particular as components of certain peptide antibiotics and in certain microorganisms. D-Amino acids have been extensively used for pharmaceutical intermediates, and as key components in many biologically important compounds including beta-lactam antibiotics, fertility drugs, anticoagulants and pesticides. Ampicillin (containing D-phenylglycine) is currently produced a scale >5000 tons per year.

### D-aminoacid Dehydrogenase (DAADH))/BspFDH1 Coupled Synthesis of 2-Aminooctanoic Acid (8)

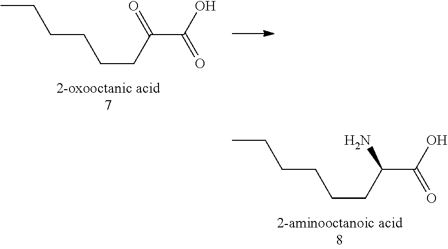

To demonstrate NADPH regeneration following synthesis of 2-aminooctanoic acid, 2-oxooctanic acid (0.5 M, 500 μL, aq) was added into potassium phosphate buffer (100 mM, pH 7.6, 2 mL) containing sodium formate (4M, 1 mL) and bovine serum albumin (100 mg/mL, 300 μL) and the reaction mixture was shaken for 3 min and then the reaction mixture was heated at 37° C. water bath for 3 minutes. NADP⁺ (30 mM, 250 μL) and BspFDH2 (17 mg/mL, 300 μL) were added and the reaction shaken for 30 minutes at room temperature. After 30 minutes, ammonium hydroxide (4 M, 1 mL) and DAADH (30 mg/mL, 200 μL) were then added. After 15 hr, t.l.c. (isopropanol/H₂O/1M NH₄OH, 8:1:1) indicated the formation of a major product (Rf 0.47). The reaction mixture was acidified by Dowex 50WX8-H+ resin until pH˜2. The resin was collected, washed with water several times to remove impurities, and then the product was eluted from the resin by ammonia solution (2M). The combined elution was concentrated in vacuo to afford D-2-aminooxtanoic acid (pale yellow powder, 29 mg, 73%). mp 256-258° C., [α]D−18.6 (c=0.76, 1M HCl), [Lit. for enantiomer: [α]D+16.0 (c=0.3, 1N HCl); Lit. for enantiomer: [α]D+21 (c=0.3, 1N HCl)]. IR,vₘₐₓ (KBr disc), 3400 (shoulder, NH₂), 2954 (br), {1659, 1622 and 1606 (s) C═O}. mp: 178-185° C. ¹H-NMR (400 MHz, D₂O-2d): δ ppm 0.74-0.85 (m, 3H), 1.15-1.34 (m, 8H), 1.67-1.88 (m, 2H), 3.65 (t, J=6.06, 1H). ¹³C-NMR (500 MHz, MeOH-4-d): δppm 14.46, 23.17, 25.61, 29.47, 31.85, 32.06, 56.13, 175.71. m/z (ES⁺) Calcd for C₈H₁₇NO₂ [M]⁺=159, C₈H₁₆NO₂ [M−H]⁺=158. Found: 159 (M)⁺, 158 (M−H)−. HRMS (ES)⁺ Cacld for C₈H₁₈NO₂ [M+H]⁺=160.1338. Found: 160.1332.

### Methods

### Nomenclature

The protein nomenclature adopted serves to identify the location of key chemical tags rather than to fully map all mutations. The use of * (eg in PSGL*-lacZ) denotes the fucosylated, sLex-modified variant.

### General Experimental

All chemicals were purchased from Sigma-Aldrich. A protease inhibitor mixture containing 4-(2-aminoethyl)benzenesulfonyl fluoride, bestatin, pepstatin A, trans-epoxysuccinyl-L-leucylamido(4-guanido)butane, and N-(α-rhamnopyranosyloxyhydroxyphosphinyl)-Leu-Trp-(phosphoramidon) was supplied by Sigma-Aldrich. Restriction endonucleases, T4 DNA ligase, Vent DNA polymerase were from New England Biolabs (Beverly, Mass.). The products of reactions were fully characterized and where appropriate also by direct comparison with commercially obtained standard.

DNA Manipulations (Heterologous Gene Expression in E. coli (pET23b))

Standard DNA procedures were used throughout (Sambrook, Molecular Ccloning Laboratory Manual) The FDH genes from Burkholderia sp were engineered for overexpression in E. coli by using the pET23b system (Novagen), which allows installation of a C-terminal H isTag sequence for protein purification.

Target FDH genes were amplified by PCR with NdeI and XhoI restriction sites incorporated. The stop codons of each FDH gene was deleted to allow C-terminal His-tag incorporation. Then amplified genes were cloned into the expression vector. The forward and reverse primers for the amplification of BcenFDH1 (Bpc184FDH) were, respectively:

(SEQ ID NO: 20)
5-CGATGTCCATATGGCTACCGTCCTGTGCG-3′
and
(SEQ ID NO: 21)
5′-CACCTCGAGCGTCAGCCGGTACGACTG-3′

The forward and reverse primers for the amplification of BspFDH2 (Bsp383-FDH) were, respectively,

(SEQ ID NO: 22)
5-CGATGTCCATATGGCCACCGTCCTGTGCG-3′
and
(SEQ ID NO: 23)
5′-CACCTCGAGTGTCAGCCGGTACGACTG -3′.

The PCR conditions consisted of 29 cycles, with denaturation at 94° C. for 50 s, annealing at 50° C. for 1 min and extension for 5 min at 72° C. The initial denaturation step was at 98° C. for 3 min. The PCR mix included PfuTurbo DNA polymerase (Stratagene) (2.5 U), 10×Pfu reaction buffer (4 μl) each dNTPs (10 mM, 1 μl), template DNA, forward and reverse primers, (100 pmol/μl, 5 μl each) in a reaction volume of 50 μl with deionised water. The PCR product was isolated and digested with NdeI and XhoI restriction endonucleases. The digested DNAs were purified and cloned into the NdeI and XhoI sites of the pET23b vector to give the final expression constructs of pET23b-Bsp184FDH and pET23b-Bsp383FDH.

### Transforming Chemically Competent Cells

All transformations were carried out using the protocols recommended by the manufacturer. Briefly, to 50 μL of competent cells Top10 (Invitrogen™) or BL21(DE3)pLysS (Novagen™) thawed on ice was added 5 μl of each ligation reaction directly. Each vial was then mixed by tapping gently, incubated on ice for 30 minutes, and then incubated in the 42° C. water bath for 30 seconds. The vials were then removed and placed on ice, 250 μL of pre-warmed SOC medium was added to each vial and incubated at 37° C. for an hour at 225 rpm in the shaking incubator. Each transformation (20-200 μL) was then spread on LB-agar plates with appropriate antibiotics (for pET23b, Amp at 100 μg/ml was used) and incubated in a 37° C. incubator overnight (10-16 hours). Occasionally, an alternative procedure was used for a rapid transformation. This procedure is only suitable for ampicillin selection. Briefly, to 50 μL of competent cells thawed on ice were added 5 μl of each ligation reaction directly. Each vial then was mixed by tapping gently and incubated on ice for 5 min. Each transformation was then spread on LB-agar plates (pre-warmed) with appropriate antibiotics (for pET23b Amp at 100 μg/ml was used) and incubated in the 37° C. incubator overnight (10-16 hours).

### Restriction Analysis (NdeI-XhoI)

Restriction analyses were performed using NdeI and XhoI endonuclease enzymes (New England BioLab). Reaction mixtures were as follows: 10 μL of plasmid purified from 1 ml of overnight culture, 2 μL BamHI buffer, 6 μL of dH₂O containing BSA (final concentration: 1×) and 1 μL of NdeI (2.5 U) and 1 μL XhoI (2.5 U) endonucleases to the total volume of 20 μL. The reaction mixture was thoroughly mixed, briefly centrifuged and incubated at 37° C. for 1 hour and 45 minutes.

### DNA Sequencing

All sequencing reactions were carried out using Applied Biosystems Big Dye™ terminators (Applied Biosystems™ 3730x1 DNA Analyzer). All sequencing was carried out on both strands. DNA sequences were determined using T7 promoter and T7 terminator primers. Nucleotide sequence data were assembled and analyzed using the BioEdit software packages.

Production of C-Terminal His-Tagged BcenFDH1 (from pET23b-Bsp184FDH) and BspFDH2 (from pET23b-Bsp383FDH)

E. coli BL21(DE3)plysS containing the named expression constructs were grown in LB medium containing 100 μg/mL ampicillin and 34 μg/mL chloroamphenicol at 37° C. After induction with 1 mM IPTG at an optical density (OD600) of 0.6, growth was continued for up to 4 h at 30° C. before harvesting. Aliquots were withdrawn at regular time points. Expression was assessed by comparing the banding pattern obtained by SDS-PAGE analysis of whole cell extracts with that of a negative control (i.e. E. coli BL21(DE3) pET23b). The solubility of the protein was assessed by a standard procedure using SDS-PAGE analysis.

### SDS-PAGE Analysis

SDS-PAGE analysis was carried out in a SDS-10-20% polyacrylamide gel (SDS polyacrylamide gel system, Gibco BRL Life Technologies, Gaithersburg, Md.) with a running buffer containing 49 mM Tris, 384 mM glycine, and 0.1% (w/v) SDS, pH 8.5). Coomassie blue staining was used for detection of the polypeptides.

FIG. 14 shows the construction of the expression vector and SDS-PAGE analysis of the expressed protein and purification of Bsp383FDH from E. coli culture BL21(DE3)plysS containing pET23b-Bsp383FDH plasmid following induction with IPTG. Lanes are as follows: 1 insoluble fraction; 2 soluble fraction (cell free extract); 3 FPLC wash; 4 loosely bound protein on H isTag column; 5 purified protein fraction; M molecular mass protein markers.

### Protein Purification

Approximately 3 g of wet cell paste was suspended in 10 mL of ice-cold buffer A (20 mM Tris-HCl, pH 7.8, 0.5 M NaCl, 5 mM imidazole) containing 10 μL of DNAese and 100 μL of protease inhibitor mixture (Sigma-Aldrichl. The cells were disrupted by sonication using a Soniprep™ 150 sonicator (Sanyo, Tokyo, Japan) fitted with a 3-mm diameter probe. The cell suspension was kept on ice and sonicated with a 15 s burst followed by a 45 s interval. This process was repeated 6 times. The resulting cell extract was centrifuged at 28,000×g for 30 min at 4° C. The cell-free extract was carefully removed and filtered through a 0.45-micron filter unit (Sartorius™, Goettingen, Germany). The clarified extract was then loaded onto a 1-ml Nickel HiTrap column (Amersham Biosciences™) equilibrated in buffer A at a flow rate of 1 mL/min. Column chromatography was performed at 4° C. The column was then extensively washed with buffer A (12 column volumes) to remove unbound material. The majority of contaminating proteins were then removed by washing with 5 mM imidazole in buffer A (10 column volumes) before elution of the recombinant His-tagged FDH by 300 mM imidazole in buffer A. The buffer was exchanged using a PD-10 desalting column after concentration (Amersham Biosciences) which was equilibrated in Buffer B (50 mM Tris-HCl, pH 7.8) at 4° C. The sample volume (4 mL) was reduced to 1 mL using a spin concentrator with a 30 kDa cut-off membrane (Sartorius). Storage of sample was achieved either aliquoted and stored frozen at −70° C. after quick-freeze in freezer-proof tubes in an acetone-dry ice or liquid nitrogen bath or, glycerol was added to a final concentration of 50% (v/v) and the sample stored at −20° C.

### Determination of Protein Concentration Using Bradford Assay

The standard protocol was performed in microtiter plates. The dye reagent (Protein Assay Dye Reagent Concentrate, Catalog No: 500-0006, Bio-Rad™) was prepared by diluting 1 part dye reagent concentrate with 4 parts double distilled water. This diluted reagent may be used for about 2 weeks when kept at room temperature. Eight dilutions of protein standards were prepared (0.00, 0.05, 0.10, 0.15, 0.25, 0.30, 0.40 and 0.50 mg/ml) that were representative of the protein solution to be tested. Protein solutions were assayed in triplicate. Each standard and sample solution (10 μL) was pipetted into separate microtiter plate wells. The diluted dye reagent (200 μL) was added to each well. The samples and reagent were mixed thoroughly using the microplate mixer and incubated at room temperature for at least 5 minutes. Absorbance was measured at 595 nm using Microtiter Plate Reader (Spectra Max Plus™, Molecular Devices). The protein concentrations in the samples were calculated using the calibration curve produced with the standards.

### Determination of Protein Concentration Using Spectroscopic Assay

Spectroscopic determination of the protein concentration of the FDHs was carried out using the reported extinction coefficient value of 1.32×10⁵ M⁻¹ cm⁻¹ at 280 nm for PseFDH. To 950 μL of sodium phosphate buffer, 0.1 M, pH 7.0 was added 50 μL of protein sample in a quartz cuvette. Absorbance was measured at 280 nm.

### Protein Mass Spectrometry-Liquid Chromatography/Mass Spectrometry

Protein samples of BcenFDH1 and BspFDH2 (Bsp184, and 383 FDHs) were introduced into the ion source as HPLC effluent. Electrospray ionization (positive mode) gave multiple peaks for the protein and MaxEnt deconvolution algorithms were used to calculate a true mass of the protein using MassLynx Software. Isotopically averaged theoretical molecular weight for BcenFDH1 without N-terminus methionine was 42462 Da; the experimentally determined averaged molecular weight for Bsp184-FDH was found to be 42462 Da Similarly, isotopically averaged theoretical molecular weight for BspFDH2 without N-terminus methionine is 42466 Da; the experimentally determined averaged molecular weight for BspFDH2 was found to be 42469 Da.

LC-MS was performed on a Micromass LCT (ESI(+)-TOF-MS) coupled to a water Alliance 2790 HPLC using a Phenomenex Jupiter C4 column (240 cm×4.6 mm×5 microm). See FIG. 15 for LCMS analysis of BcenFDH1 and BspFDH2. Water (solvent A) and acetonitrile (solvent B), each containing 0.5% formic acid, were used as the mobile phase at a flow rate of 1 ml/min. The gradient was programmed as follows: 95% A (3 min isocratic) to 100% B after 16 min the isocratic for 2 min. The electrospray source of the LCT was operated with a capillary voltage of 3 kV and cone voltage of 30V. Nitrogen was used as the nebuliser and desolvation gas at a total flow 400 L/h. Myoglobin (horse heart) was used as a calibration standard and to test the sensitivity of the system. FIGS. 5 and 6, respectively, show the mass spectrometric analysis of BcenFDH1 (Bsp184FDH) (FIG. 5 (a) chromatogram (b) multiple charge state ‘RAW’ spectrum (c) MaxEnt deconvoluted spectrum) and of BspFDH2 (Bsp383FDH) (FIG. 6(a) chromatogram (b) multiple charge state ‘RAW’ spectrum (c) MaxEnt deconvoluted spectrum).

### Assay of Enzyme Activity

Enzyme activity was routinely measured spectrophotometrically using a Microtiter Plate Reader (SpectraMax Plus™, Molecular Devices) by monitoring the increase of NADH concentration (λ₃₄₀=6.22 mM⁻¹ cm⁻¹) in 0.1 M sodium phosphate buffer (pH 7.0) containing 5 mM EDTA, 200 mM sodium formate and 1 mM NAD at 37° C. The reaction was initiated by addition of NAD⁺. The total volume of the reaction mixture was 1 mL. Specific activity (U/mg enzyme) was defined as number of mols of product formed per mg enzyme per minute.

### Steady State Kinetics for Formate Oxidation

BcenFDH1 (Bsp184FDH) and BspFDH2 (Bsp383FDH) catalyzed oxidation of formate to CO₂ was monitored by UV/Vis spectrophotometry in a 96-well plate format. A series of eight solutions of formate (0-200 mM) were prepared by successive dilutions of a stock solution of 0.5 M sodium formate in 0.1 M sodium phosphate buffer, pH 7.0. From this series, 170 μL was transferred into wells of the 96-well plate. 10 μL of the FDHs from a stock solution (64.1 μM for BcenFDH1 (Bsp184FDH) and 78.0 μM for BspFDH2 (Bsp383FDH)) were added into each well and equilibrated for 3 min at 30° C. The reaction was initiated by addition of 20 μL of NAD⁺ or NADP⁺ solution from a freshly prepared stock (10 mM) using an 8-channel pipette. The plate was then immediately placed into the plate reader. The plate was shaken for 5 s to ensure thorough mixing, and then time-based measurements were recorded every 15 s for 10 min. The rate of formate oxidation was calculated using an extinction coefficient of 3.38 mM⁻¹ (for 200 μL), which was determined using NADH standards. Kₘ and Vₘₐₓ values were obtained by non-linear regression to the Michaelis-Menten equation (Eq.1 below) using Origin v7 (Microcal Software Inc.).

v=VₘₐₓS/(Kₘ+S) (1)

### Steady State Kinetics for NAD⁺ and NADP⁺

Similar procedures were used for determination of Michaelis-Menten parameters for NAD⁺ and NADP⁺. A series of eight solutions of cofactor (0-75 mM) were prepared by successive dilutions of stock solutions. 150 μL of sodium formate solutions (180 mM in well) were transferred into wells of the 96-well plate. 10 μL of the FDHs from a stock solution (64.1 μM for BcenFDH1 (Bsp184FDH) and 78.0 μM for BspFDH2 (Bsp383FDH)) were added into each well and equilibrated for 3 min at 30° C. The reaction was initiated by addition of 20 μL of NAD⁺ or NADP⁺ solutions using an 8-channel pipette. The plate was then immediately placed into the plate reader. The plate was shaken for 5 s to ensure thorough mixing, and then time-based measurements were recorded every 15 s for 10 min. The rate of formate oxidation was calculated using an extinction coefficient of 3.38 mM⁻¹ (for 200 μL), which was determined using NADH standards. Kₘ and Vₘₐₓ values were obtained by non-linear regression to the Michaelis-Menten equation as before.

### Inhibition Studies of Bsp383FDH or Bsp184FDH

Inhibition of formate oxidation by Bsp383FDH or Bsp184FDH was performed in the presence of the following potential inhibitors: NaN₃, NaNO₂, KNO₃, KNO₂, KCN and iodoacetamide at 5 mM and 20 mM concentrations. In a typical procedure, 10 μL of enzyme, Bsp383FDH (0.022 mg/mL) or Bsp184FDH (0.020 mg/mL) was incubated in sodium phosphate buffer (50 mM, pH 7.0) containing 100 mM of formate (sodium formate) and 5 mM and 20 mM inhibitors concentrations for 5 minutes at room temperature. Reaction of formate oxidation was then initiated by addition of NADP in 5 mM final concentration. Absorbance at 340 nm due to NADPH formation was spectrophotometrically monitored using a microtiter plate reader. Absorbance was recorded at 10 seconds intervals for 10 minutes. Initial activities were then calculated using the linear part of the initial readings using at least 20 data points and used directly as relative activity values. Control experiments were also performed in the absence of the enzyme.

FIG. 11 shows the inhibition effects of several anions on Bsp184FDH(BcenFDH1)-catalysed formate oxidation. FIG. 12 shows the inhibition effects of several anions on Bsp383FDH(BspFDH2)-catalysed formate oxidation.

### pH Profile Determination

The pH profiles for BcenFDH1 (Bsp184FDH) and BspFDH2 (Bsp383FDH) catalyzed formate oxidation, shown in FIGS. 13(a) and (b), were investigated by Uv/Vis spectrophotometry in a 96-well plate format. 8×120 mM of sodium formate solutions were prepared in a series of eight buffer solutions of sodium phosphate with pH from 5.85 to 8.0 (50 mM), which were prepared by successive mixing of mono and dibasic sodium phosphate stock solution. From this series, 170 μL was transferred into wells of the 96-well plate. 10 μL of the FDHs from a stock solution (64.1 μM for BcenFDH1 (Bsp184FDH) and 78.0 μM for BspFDH2 (Bsp383FDH)) were added into each well and equilibrated for 3 min at 30° C. The reaction was initiated by addition of 20 μL of NAD⁺ solution from a freshly prepared stock (10 mM) using an 8-channel pipette. The plate was then immediately placed into the plate reader. The plate was shaken for 5 s to ensure thorough mixing, and then time-based measurements were recorded every 15 s for 10 min. The rate of formate oxidation was calculated using an extinction coefficient of 3.38 mM⁻¹ (for 200 μL). FIG. 13 shows the operative pH Range of the purified (a) BcenFDH1 and (b) BspFDH2 towards the oxidation of formate to carbon dioxide.

### Thermal Stability Study

Thermal stability for purified wild-type BcenFDH1 (Bsp184FDH) and BspFDH2 (Bsp383FDH) catalyzed formate oxidation were investigated by Uv/Vis spectrophotometry in a 96-well plate format. The results are illustrated in FIGS. 19 and 20. 50 μL of BcenFDH1 (2.1 mg/mL) and BspFDH2 (1.5 mg/mL) were incubated individually at temperature in the range of 0° C. to 80° C. (0° C., 37° C., 45° C., 50° C., 55° C., 60° C., 70° C., and 80° C.) for 20 minutes. In addition, time based-thermal stability for purified wild-type BcenFDH1 (Bsp184FDH) and BspFDH2 (Bsp383FDH) catalyzed formate oxidation were also investigated at 60° C. and 70° C. over 48 hours in a similar way. The samples were spun down briefly to remove any precipitations before use. 10 μL of heat-treated BcenFDH1 and BspFDH2 were added into each well and equilibrated for 3 min at 30° C. after adding 170 μL of 200 mM sodium formate (dissolving in 50 mM sodium phosphate buffer, pH 7.0.) The reaction was initiated by addition of 20 μL of NAM⁺ solution from a freshly prepared stock of 20 mM using an 8-channel pipette. The plate was then immediately placed into the plate reader. The plate was shaken for 5 seconds to ensure thorough mixing. Time-based measurements were recorded every 10 s for 10 min. The rate of formate oxidation was calculated using the absorption coefficient of 3.38 mM⁻¹ (for 200 μL).

Up to 65° C., there is no activity loss for either of the FDHs. Above this temperature, Bsp184FDH started to lose activity with complete loss of activity at about 70° C. For Bsp383FDH, about 80% activity is retained at 75° C.

The results show that both of these tested FDHs have comparable or improved stability with the reported NAD-dependent FDHs in the literature, as set out in Table A below.

TABLE A

| | Incubation | Temperature, | Remaining | |
|---|---|---|---|---|
| Enzymes | Time, min | ° C. | activity | References |
| FDH from | 60 | 50 | 100% | Shinoda, |
| Paracoccus sp. 12-A | | 55 | 25% | 2002 |
| FDH from | 10 | 50 | 100% | Nanba |
| Ancylobacter | | 55 | 60% | 2003b |
| acquaticus, | | 60 | 0% | |
| Strain KNK607M | | | | |
| FDH from | 10 | 50 | 90% | Nanba |
| Thiobacillus sp | | 55 | 50% | 2003a |
| Strain KNK65MA | | 60 & 70 | 0% | |
| FDH from | 10 | 50 | 100% | Asano 1988 |
| Moraxella sp | | 55 | 50% | |
| FDH from | 60 | 50 | 100% | Lida, 1992 |
| Paracoccus sp. 12-A | | 55 | 10% | |

Thiskov et al (Biomolecular Engineering, 23, 89-110 (2006)) provides a comparison of wild type as well as mutant FDHs in terms of their thermal stability expressed as Tₘ—the temperature at which there is 50% inactivation after 20 minutes. Most FDHs show Tₘ values between 52-64° C. Accordingly, Bsp184FDH exhibits comparable stability with other FDHs, whilst Bsp383FDH shows superior thermal stability, with most of its activity being retained following incubation at 75° C. for 20 minutes.

### Site-Directed Mutagenesis

An overlap extension PCR-based site directed mutagenesis method using QuickChange II XL™ site directed mutagenesis Kit (Stratagene™) was utilised to create the desired mutants for all FDHs. All experimental procedures were carried out according to the manufacturer's instructions.

A single mutation was introduced into the Candida methylica FDH gene at position 195 and Burkholderia FDH genes at position 223. The following synthetic oligonucleotides were designed to carry out the desired mutations:

D195Q-CmetFDH:
(F)
Seq ID No: 24
5-GAATTATTATACTACCAGTATCAAGCTTTACC-3′ -
(R)
Seq ID No: 25
5′-GGTAAAGCTTGATACTGGTAGTATAATAATTC-3′ -
Q223D-BspFDH:
(F)
Seq ID No: 26
5′-GCTGCACTACACGGATCGCCACCGGCTCG-3′ -
(R)
Seq ID No: 27
5′-CGAGCCGGTGGCGATCCGTGTAGTGCAGC-3′ -

### Protein Crystallization

Crystallization trials was carried out in 96 well format using so called ‘Limited Protein Screen’, Crystal screen II and PEG/Ion screen (all from Hampton research). Of the conditions that produced crystals, 170 mM sodium sulphate, 11.5% PEG3350 gave the most desirable crystal morphology. Optimization was undertaken using the vapour diffusion sitting drop method in 24 well format at 19° C. (Cryschem Plate, Hampton Research, USA). The optimization trays were set up around the original conditions of 170 mM sodium sulphate and 11.5% PEG 3350 for both native and Q223D Bsp383FDH mutant at 14 mg mL⁻¹, with a final mother liquor volume of 0.5 mL. Drops were dispensed at a protein: mother liquor ratio of 1:1 to give a final volume of 2 μL. Bipyramidal shaped crystals grew over 2-12 days.

Bsp383FDH-NADP⁺ and Q223D Bsp383FDH-NAD⁺ complex crystals were obtained by the addition of 5-20 mM of the respective cofactor to the mother liquor before the drops were dispensed. The final mother liquor volume was 0.5 mL. To obtain Bsp383FDH-NADP⁺-formate complex crystals 10 mM NADP⁺, 5 mM sodium azide and 400 mM sodium formate was added to the original crystallization conditions to give a final mother liquor volume of 0.5 mL. Complex crystals would take between 2 and 12 days to grow and had the same morphology as the native crystals. All crystals were cryoprotected in a solution consisting of the well solution with 20% ethylene glycol as the cryoprotectant before being flash frozen in liquid nitrogen.

### Structure Determination and Refinement

Initially a 1.8 Å resolution dataset was collected from a single native Bsp383FDH crystal at a temperature of 100K on beamline ID 14-1 at the European Synchrotron Radiation Facility, Grenoble (ESRF). The data set was integrated with MOSFLM, then scaled and reduced using SCALA from the CCP4 suite of programs (Collaborative Computational Project, N. The CCP4 suite: programs for protein crystallography. Acta Crystallographica Section D 50 (5), 760-763 (1994). The FDH structure was solved by molecular replacement using PHASER (Read, R. J. Acta Crystallographica Section D 57, 1373-1382 (2001)) with the protein atoms of a monomer from PDB entry 2NAC. Data was used between 60 and 4.5 Å. PHASER placed 3 molecules in the asymmetric unit (reflecting a solvent content of ˜52%). The structure was refined using iterative cycles of REFMAC (Murshudov, G. N., Vagin, A. A., & Dodson, E. J. Refinement of macromolecular structures by the maximum-likelihood method. Acta Crystallographica Section D 53, 240-255, (1997) with model building and solvent addition with COOT (Emsley, P. & Cowtan, K. Coot: model-building tools for molecular graphics. Acta Crystallographica Section D 60, 2126-2132 (2004)). A further native data set was collected to 1.8 Å, going to 1.5 Å at the edges of the square detector, from a single crystal at ID 14-4 (ESRF); data were processed using HKL2000 (Otwinowski, Z. & Minor, W. Macromolecular Crystallography, Pt A 276, 307-326 (1997).

Diffraction data to 2.5 Å resolution of the Bsp383FDH-NADP complex was collected on beamline ID29 (ESRF). The refined native Bsp383FDH coordinates were used as a molecular replacement model in PHASER to solve the Bsp383FDH-NADP complex. The NADP⁺ coordinates were obtained through PRODRG (Schuttelkopf, A. W. & Aalten, D. M. F. Acta Crystallographica Section D, 1355-1363 (2004)) and placed into the density using COOT.

Further refinement was carried out by REFMAC and solvent addition by COOT.

The Bsp383FDH-NADP-formate/azide complex data were collected from a single crystal at beamline IO4 (Diamond, Oxford) to 2.0 Å. Q223D Bsp383FDH—NAD⁺ complex data to 2.0 Å were collected at ID14-4 (ESRF). Both scaled data sets were isomorphous with the native data so the native model was used as the starting model for refinement. Stereochemical dictionaries for NAD and NADP were obtained from PRODRG and HIC-UP respectively (Schuttelkopf, A. W. & Aalten, D. M. F. Acta Crystallographica Section D, 1355-1363 (2004); Kleywegt, G. J. & Jones, T. A. Databases in protein Crystallography. Acta Crystallographica Section D 54, 1119-1131 (1998)).

Crystallisation of Burkholderia sp383 FDH

Crystallization of Bsp383 FDH in the presence of NADP⁺ was attempted to obtain a complex structure. Crystal growths were obtained in the form of square pyramidal. It appeared that in the presence of NADP⁺, crystal growth was quicker than in its absence. Optimal results were obtained with sodium sulphate and PEG3350 in the presence of NADP (5-30 mM). In contrast to previous observations for PseFDH, the presence of eight additional amino acids (Leu-Glu-6×His (SEQ ID NO: 28)) at the C-terminus of the recombinant wt-Bsp383FDH did not interfere with crystallisations, resulting in production of high quality crystals of recombinant Bsp383FDH with and without NADP or/and formate.

Initial crystallization trials was carried out in 96 well format using so called ‘Limited Protein Screen’, Crystal screen II and PEG/Ion screen (all from Hampton research). When crystals were obtained, information from initial crystal screen conditions was used to optimize crystallisation conditions to produce crystals suitable for x-ray diffraction analysis. Crystal optimizations were performed manually using the sitting drop technique using plates in 24 well formats (Cryschem Plate, Hampton Research, USA). In typical experiments for obtaining crystals of BspFDH2, 1 μL of droplet of protein solution (14 mg/mL in 50 mM Tris.HCl, pH 7.5) was mixed with 1 μL of crystallization reagent (170 mM sodium sulphate, 11.5% PEG3350) on a platform in vapour equilibration with the reagent. To obtain protein-NADP complex crystals, NADP (5-20 mM) was added to the crystallisation reagents. To obtain protein-NADP⁺-formate complex crystals NADP (10 mM), sodium azide (5 mM) and sodium formate (0.4 M) were also included in the crystallization reagents. The reservoir volume was 500 μL. The plates were sealed with clear sealing type and placed in a shelf at room temperature. Bipyramidal shaped crystals grew over 2-12 days. Crystal growths were monitored using a microscope (Nikon SMZ1000) and images were recorded on a digital net camera (Nikon, DN100). At various time intervals, crystal growths were investigated and recorded. Before data collection, FDHs' crystals were first equilibrated with the well solution before cryo-freezing. Pre-equilibrated FDH crystals was then transferred together with about 5-10 μL of the original well solution to a reservoir of 0.1 mL of cryoprotectant solution. The cryoprotectant solution contained 170 mM sodium sulphate, 11.5% PEG3350 and 20% ethylene glycol. Immediately after the first transfer, crystals were picked up in a drop using a cryo-loop that was mounted on a stainless still cap and was frozen in liquid nitrogen.

### Bsp383FDH Mutant Screen

### Experimental

Enzymatic activity from screening of 103 colonies for Q223X-Bsp383 FDH was conducted as follows. The following mutagenic oligonucleotide primers were used for saturation mutagenesis at position 223 on Bsp383 FDH (mismatched bases are highlighted): 5′-CTG CAC TAC ACG NNN CGC CAC CGG CTC G-3′ and 5′-C GAG CCG GTG GCG NNN CGT GTA GTG CAG-3′ (N=A, T, G and C). The mutagenesis was conducted by GenScript Ltd. The mix of mutated plasmids and WT plasmid were transformed into BL21(DE3) competent cells for protein expression. Colonies grown overnight were inoculated in 1 ml cultures of Autoinduction media (Novagen) supplemented with Ampicillin (final conc. 100 μl/mL) in 96-well deep-well plates (Abgene). The plates were covered with gas permeable films (Abgene) and incubated at 37° C. with shaking at 300 rpm overnight. 100 μl samples from the overnight grown cultures were mixed with 50 μl of sterile 50% glycerol and stored at −80° C. The remaining culture volume was lysed with 50 μl of BugBuster Master Mix reagent (Novagen) at room temperature for 30 min with shaking at 300 rpm. The resulting lysate was clarified by centrifugation at 3750 rpm for 10 min. The clear supernatant was transferred to new plate and used to determine enzymatic activity in Microtiter Plate Reader (Spectra Max plus, Molecular Devices). 15 μl of cell lysis was premixed with sodium formate (100 mM, 165 μl) and NAD⁺/or NADP⁺ (10 mM, 20 μl). After incubation for 3 minutes, the Vmax was measured by 96 well UV/Vis microplate reader (measured λ=340 nm). Vmax of WT Bsp383FDH was around 45 ΔAbs/min.

BL21 (DE3) E. coli cells harboring WT Bsp393 FDH and pET 23b expression vector were used as a negative control. Autoinduction media was used to avoid the need for IPTG addition and OD₆₀₀ measurement. Commercial reagent BugMaster Mix used for cell lysis did not affect the enzymatic activity screen. It was not considered necessary to normalize activity to OD₆₀₀ of cells.

DNA sequencing was carried out on selected samples; these were those with Vmax of mutated Bsp383FDHs over 20 ΔAbs/min.

The amino acid residue at position 195 on CboFDH, which is corresponding to the activity site (D223) on Bsp383FDH, was subjected to saturation mutagenesis in accordance with the paper Andreadeli et al. (FEBS J. 275, 3859-3869 (2008)).

### Results and Discussion

The results of the Vmax screening are shown in FIG. 21.

The results of DNA sequencing are summarized in Table B and Table C. In general, amino acids with relatively small, non-aromatic side-chains (Asn (N), Ala (A), Ser (S), Thr (T), Lys (K)) displayed higher dual NADP⁺ and NAD⁺ activity. However, Asp (Q) displayed the highest NAD⁺/NADP⁺ activity ratio and Gln (D) displayed the highest NADP⁺/NAD⁺ activity ratio.

TABLE B
DNA Sequencing results of selected colonies with higher NADP⁺/NAD⁺
affinity

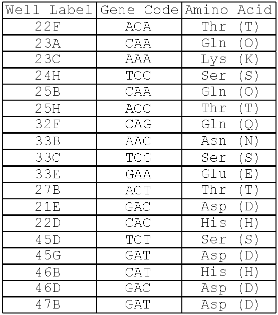

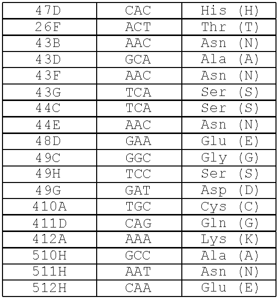

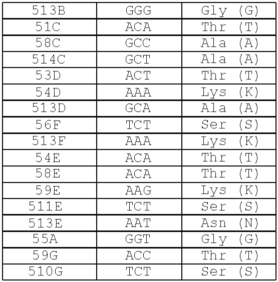

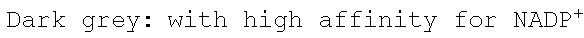

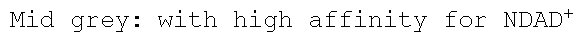

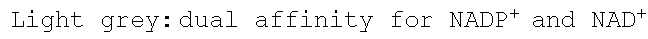

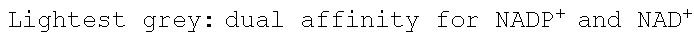

Others: lower dual affinity for NADP⁺ and NAD⁺

TABLE C
Summary of amino acids in selected colonies

| | Total number |
|---|---|
| Amino acid | present |
| Asp (D) | 5 |
| Glu (Q) | 4 |
| Asn (N) | 6 |
| Ala (A) | 5 |
| Ser (S) | 9 |
| Lys (K) | 5 |
| Thr (T) | 9 |
| His (H) | 3 |
| Cys (C) | 1 |
| Glu (F) | 3 |
| Gly (G) | 3 |

The results of the saturation mutagenesis showed that mutants Q195S, Q195N and Q195D CboFDHs exhibited higher NADP⁺/NAD⁺ activity ratio but both Q195S and A195N CboFDHs showed lower affinity for NADP⁺ compared to Q195D CboFDH. In addition, the WT CboFDH with Asp(Q) at position 195 showed the higher NAD⁺/NADP⁺ activity ratio. The screening results of this work and from the Andreadeli et al. (2008) paper confirmed that Q223 Bsp383 FDH is NADP⁺ preferred and D223 Bsp383 FDH is NAD⁺ preferred.

### Measurement of Total Turnover Number of NADP⁺

### Experimental

Sodium formate (200 mM) and NADP⁺ in different volumes and concentrations were premixed with WT Bsp383 FDH (1.4 mg/mL, 50 μL) under room temperature for 15 min. The reaction mixture was oxygenated for 5 min. Then, different volume of propylbenzene (500 mM in DMSO stock) and WT P450 BM3 (4 mg/mL, 50 μL) were added into the reaction mixture shaking under room temperature overnight. The final product was extracted by DCM and the conversion yield and total turnover number was determined by GC-MS (Rt_{—}_{_{SM}}˜3.2 minutes; Rt_product˜5.3 minutes). The GC-MS data were collected in full-scan mode (m/z 50-300) using a BPX-5 column (0.25 mm×15 m, 0.25 um film thickness) on a GCT system (GC: Agilent G890 series, MS: Micromass). The following GC program was used: 80° C. (2 min hold), 80-280° C. (10° C./min) and 280° C. (5 min hold).

### Results and Discussion

Results are shown in Table D. It can be seen that the total turnover number of NADP⁺ can be achieved around 24,087 mol product/mol NADP⁺ and conversion yield was around 75%. Compared to other published results (Kizaki N. Appl Microbiol Biotechnol 55, 590-595 (2001)), WT Bsp383 FDH can achieve higher total turnover number of NADP⁺ and retain a high conversion yield.

TABLE D
WT Bsp383 FDH total turnover number of NADP⁺

| | Rxn Label | | | | | | | | | |
|---|---|---|---|---|---|---|---|---|---|---|
| | 1 | | 2 | | 3 | | 4 | | 5 | |
| Tris-HCl | 1.145 | mL | 390 | μL | 0 | | 714 | μL | 714 | μL |
| (50 mM, pH 7.3) | | | | | | | | | | |
| Sodium Formate | 375 | μL | 750 | μL | 1.5 | mL | 750 | μL | 750 | μL |
| (200 mM) | | | | | | | | | | |
| PropylBenzene | 20 | μL | 40 | μL | 80 | μL | 40 | μL | 40 | μL |
| (500 mM in DMSO) | | | | | | | | | | |
| NADP⁺ | 360 | μL | 360 | μL | 360 | μL | 36 | μL | 36 | μL |
| | (1.74 | mM) | (1.74 | mM) | (1.74 | mM) | (1.74 | mM) | (0.174 | mM) |
| WT Bsp383 FDH | 50 | μL | 50 | μL | 50 | μL | 50 | μL | 50 | μL |
| (1.4 mg/mL) | | | | | | | | | | |
| WT P450 BM3 | 50 | μL | 50 | μL | 50 | μL | 50 | μL | 50 | μL |
| (4 mg/mL) | | | | | | | | | | |

| | | | | | |
|---|---|---|---|---|---|
| GC conversion yield (%) | >99 | >99 | >99 | >99 | >99 |
| Total Turnover Number | 16 | 32 | 64 | 320 | 3200 |
| (mol product/mol NADP⁺) | | | | | |
| Total Turnover Number | 6050 | 12101 | 24202 | 12101 | 12101 |
| (mol product/mol enzyme-FDH) | | | | | |

| | Rxn Label | | | | | | | |
|---|---|---|---|---|---|---|---|---|
| | 6 | | 7 | | 8 | | 9 | |
| Tris-HCl | 714 | μL | 714 | μL | 714 | μL | 714 | μL |
| (50 mM, pH 7.3) | | | | | | | | |
| Sodium Formate | 750 | μL | 750 | μL | 750 | μL | 750 | μL |
| (200 mM) | | | | | | | | |
| PropylBenzene | 40 | μL | 40 | μL | 40 | μL | 40 | μL |
| (500 mM in DMSO) | | | | | | | | |
| NADP⁺ | 36 | μL | 36 | μL | 36 | μL | 36 | μL |
| | (0.087 | mM) | (0.043 | mM) | (0.029 | mM) | (17.4 | μM) |
| WT Bsp383 FDH | 50 | μL | 50 | μL | 50 | μL | 50 | μL |
| (1.4 mg/mL) | | | | | | | | |
| WT P450 BM3 | 50 | μL | 50 | μL | 50 | μL | 50 | μL |
| (4 mg/mL) | | | | | | | | |

| | | | | |
|---|---|---|---|---|
| GC conversion yield (%) | ~83 | ~83 | ~70 | ~75 |
| Total Turnover Number | 5290 | 10727 | 13415 | 24087 |
| (mol product/mol NADP⁺) | | | | |
| Total Turnover Number | 10044 | 10044 | 8471 | 9076 |
| (mol product/mol enzyme-FDH) | | | | |

### Measurement of Total Turnover Number of WT Bsp383FDH

### Experimental

The same methods were used as in the experimental described above for measurement of the total turnover number of NADP⁺. Starting from the conditions under which the best TTN of NADP⁺ was achieved, the concentration of enzyme was decreased slowly. Both TTN of NADP+ and TTN of enzyme were determined.

### Results and Discussion

Results are shown in Table E. The best TTN of NADP⁺ and enzyme in one complete reaction is the reaction labeled E-3. Under these conditions both TTN of NADP+ and TTN of enzyme are achieved in the efficient range of 10⁴-10⁵. A second reaction is shown labeled as E-4 under which both TTN of NADP+ and TTN of enzyme are efficient. It can be seen that as compared to E-3 although the enzyme turnover is higher, the NADP+ turnover is lower.

TABLE E
Total turnover number of WT Bsp383 FDH, NADP⁺ as cofactor

| Rxn Label | E-3 | E-4 |
|---|---|---|
| Tris-HCl | 307 μL | 357 μL |
| (50 mM, pH 7.3) | | |
| Sodium Formate | 375 μL | 375 μL |
| (200 mM) | | |
| PropylBenzene | 20 μL | 20 μL |
| (500 mM in DMSO) | | |
| NADP⁺ | 18 μL (0.026 mM) | 18 μL (0.026 mM) |
| WT Bsp383 FDH | 75 μL | 25 μL |
| (0.2 mg/mL) | | |
| WT P450 BM3 | 25 μL | 25 μL |
| (4 mg/mL) | | |
| GC conversion yield (%) | >99 | ~70 |
| Total Turnover Number | 21367 | 14956 |
| (mol product/mol NADP⁺) | | |
| Total Turnover Number | 28236 | 59297 |
| (mol product/mol enzyme-FDH) | | |

### Comparison of Total Turnover Number of WT Bsp383FDH and PTDH(Codexis)

### Experimental

Similar methods were used as in the experimental described above for measurement of the total turnover number of NADP⁺ to compare the TTN of WT Bsp383FDH and the TTN of PTDH(Codexis).

### Results

The results are shown in Table F.

TABLE F
Comparison of total turnover number of WT Bsp383FDH and PTDH (from Codexis),
WT P450 BM3 as the coupled enzyme

| Rxn Label | FDH/BM3-1 | FDH/BM3-2 | PTDH/BM3-1 | PTDH/BM3-2 |
|---|---|---|---|---|
| Tris-HCl | 307 μL | 357 μL | 215 μL | 252.5 μL |
| (50 mM, pH 7.3) | | | | |
| Sodium Formate | 375 μL | 375 μL | — | — |
| (200 mM) | | | | |
| Phosphite solution | — | — | 600 μL | 600 μL |
| (200 mM) | | | | |
| PropylBenzene | 20 μL | 20 μL | 40 μL | 40 μL |
| (500 mM in DMSO) | | | | |
| ethyl 4-chloro-3-oxobutanoate | — | — | — | — |
| (500 mM in DMSO) | | | | |
| NADP⁺ | 18 μL (0.026 mM) | 18 μL (0.026 mM) | 20 μL (0.026 mM) | 20 μL (0.026 mM) |
| PTDH (From Codexis) | — | — | 75 μL | 37.5 μL |
| (0.2 mg/mL) | | | | |
| WT Bsp383 FDH | 75 μL | 25 μL | — | — |
| (0.2 mg/mL) | | | | |
| WT P450 BM3 | 25 μL | 25 μL | 50 μL | 50 μL |
| (4 mg/mL) | | | | |
| KRED-1 (2 mg/mL) | — | — | — | — |
| GC conversion yield (%) | >99 | ~70 | >99 | >99 |
| Total Turnover Number | 21367 | 14956 | 38461 | 38461 |
| (mol product/mol NADP⁺) | | | | |
| Total Turnover Number | 28236 | 59297 | 46512* | 93024* |
| (mol product/mol enzyme-FDH) | | | | |

*Molecular weight of PTDH was estimated by 12% SDS-PAGE gel (~25 KDa)

Comparison of Total Turnover Number of WT Bsp383FDH and PTDH(Codexis), with KRED-1, NADP⁺ Specific (from Codexis) as the Coupled Enzyme

### Experimental

Similar methods were used as in the experimental described above for measurement of the total turnover number of NADP⁺ to compare the TTN of WT Bsp383FDH and the TTN of PTDH(Codexis), with KRED-1, NADP⁺ specific (from Codexis) as the coupled enzyme.

### Results

The results are shown in Table G.

TABLE G
Comparison of total turnover number of WT Bsp383FDH and PTDH (from Codexis),
KRED-1, NADP⁺ specific (from Codexis) as the coupled enzyme

| Rxn Label | FDH/KRED-1 | FDH/KRED-2 | PTHD/KRED-1 | PTDH/KRED-2 |
|---|---|---|---|---|
| Tris-HCl | 215 μL | 252.5 μL | 215 μL | 252.5 μL |
| (50 mM, pH 7.3) | | | | |
| Sodium Formate | 600 μL | 600 μL | — | — |
| (200 mM) | | | | |
| Phosphite solution | — | — | 600 μL | 600 μL |
| (200 mM) | | | | |
| PropylBenzene | — | — | — | — |
| (500 mM in DMSO) | | | | |
| ethyl 4-chloro-3-oxobutanoate | 40 μL | 40 μL | 40 μL | 40 μL |
| (500 mM in DMSO) | | | | |
| NADP⁺ | 20 μL (0.026 mM) | 20 μL (0.026 mM) | 20 μL (0.026 mM) | 20 μL (0.026 mM) |
| PTDH (From Codexis) | — | — | 75 μL | 37.5 μL |
| (0.2 mg/mL) | | | | |
| WT Bsp383 FDH | 75 μL | 37.5 μL | | |
| (0.2 mg/mL) | | | | |
| WT P450 BM3 | — | — | — | — |
| (4 mg/mL) | | | | |
| KRED-1 (2 mg/mL) | 50 μL | 50 μL | 50 μL | 50 μL |
| GC conversion yield (%) | ~32 | ~25 | 0 | 0 |
| Total Turnover Number | 12307 | 9615 | 0 | 0 |
| (mol product/mol NADP⁺) | | | | |
| Total Turnover Number | 18156 | 28236 | 0 | 0 |
| (mol product/mol enzyme-FDH) | | | | |

1. An isolated, synthetic or recombinant formate dehydrogenase (FDH) polypeptide specific for NADP⁺ comprising:
(a) an amino acid sequence having at least about 80% sequence identity with SEQ ID NO:1 or SEQ ID NO:2, wherein the polypeptide has formate dehydrogenase (FDH) activity specific for an NADP⁺;
(b) the polypeptide of (a) wherein polypeptide has an adenine ribose recognition loop comprising an amino acid sequence having at least about 85% identity to the sequence of amino acid residues 222 to 227 or 222 to 228 in SEQ ID NO:1 or SEQ ID NO:2;
(c) a polypeptide having an adenine ribose recognition loop comprising an amino acid sequence having at least about 85% identity to the sequence of amino acid residues 222 to 227 or 222 to 228 in SEQ ID NO:1 or SEQ ID NO:2, wherein the polypeptide has formate dehydrogenase (FDH) activity specific for an NADP⁺;
(d) the polypeptide of (a), (b) or (c), wherein the polypeptide exhibits a preference for NADP⁺ over NAD⁺ of greater than 10 fold based on (kcat/Km)^{NADP+}/(kcat/Km)^{NAD+}; or
(e) the polypeptide of any of (a) to (d), wherein polypeptide is a modified wild type polypeptide, or the polypeptide is a wild type polypeptide.

2-3. (canceled)

4. The isolated, synthetic or recombinant formate dehydrogenase (FDH) polypeptide of claim 1, wherein the polypeptide has an amino acid sequence of SEQ ID No: 1 or SEQ ID No: 2.

5. The isolated, synthetic or recombinant formate dehydrogenase (FDH) polypeptide of claim 1, wherein the polypeptide comprises an adenine ribose recognition loop which comprises an amino acid sequence having at least about 85% identity to the sequence of amino acids 222 to 227 and 222 to 228 in SEQ ID No: 1 or SEQ ID No:2.

6. The isolated, synthetic or recombinant formate dehydrogenase (FDH) polypeptide of claim 1, wherein the polypeptide comprises an adenine ribose recognition loop which comprises the amino acid sequence of amino acids 222 to 227 or 222 to 228 in SEQ ID No: 1 or SEQ ID No:2.

7. The isolated, synthetic or recombinant FDH polypeptide of claim 1, wherein:
(a) the polypeptide comprises an adenine ribose recognition loop comprising a first large amino acid and a second amino acid, wherein the first and second amino acid are arranged in space to allow the second amino acid to bond with a phosphate group
(b) the polypeptide of (a), wherein the second amino acid is arranged in space to allow the amino acid to bond with a phosphate of NADP⁺;
(c) the polypeptide catalyzes one or both of the conversion of NADP⁺ to NADPH and the conversion of NAD⁺ to NADH;
(d) the polypeptide of (a), (b) or (c), wherein the first large amino acid has a van der Waals volume at least about 110 A°3/molecule;
(e) the polypeptide of any of (a) to (d), wherein the first large amino acid is selected from the group consisting of glutamine, tyrosine, phenylalanine, methionine, isoleucine, leucine, lysine, tryptophan, histidine and arginine;
(f) the polypeptide of any of (a) to (e), wherein the second amino acid is selected from the group consisting of arginine, lysine, glutamic acid, glutamine and aspartic acid;
(g) the polypeptide of any of (a) to (f), wherein the first large amino acid comprises glutamine and the second amino acid comprises arginine;
(h) the polypeptide of any of (a) to (g), wherein the first large amino acid is adjacent to the second amino acid;
(i) the polypeptide of any of (a) to (h), wherein the folded functional polypeptide the first large amino acid and the second amino acid are no more than about 10 angstroms apart; or
(j) the polypeptide of any of (a) to (i), wherein the first large amino acid is at a position corresponding to amino acid 223 in either SEQ ID NO: 1 or SEQ ID NO: 2, and the second amino acid is at a position corresponding to amino acid 224 in either SEQ ID NO: 1 or SEQ ID NO: 2.

8-19. (canceled)

20. An isolated, synthetic or recombinant polynucleotide encoding a polypeptide of claim 1.

21. The isolated, synthetic or recombinant of claim 1, wherein the adenine ribose recognition loop has been mutated at least one position to alter the three dimensional polypeptide structure of the adenine ribose recognition loop to allow a phosphate group to be recognised.

22. A method of preparing a modified FDH polypeptide which recognizes NADP⁺ comprising:
a) providing a NAD⁺ specific FDH polypeptide having an amino acid sequence with at least 50% sequence identity to the sequence of one or more of SEQ ID NOs: 3 to 19;
b) selecting an amino acid residue in the NAD⁺ specific FDH polypeptide corresponding to amino acid position 223 in SEQ ID NO: 1 or SEQ ID NO: 2;
c) providing an alternative amino acid at the position selected in b) to that which occurs in a);
d) preparing a modified polypeptide with the sequence of c); and
e) selecting a modified polypeptide prepared in d) which can recognize NADP⁺.

23. The method of claim 22, wherein in step c) the amino acid is changed to a glutamine.

24. The method of claim 22 wherein the modified polypeptide of step d) is at least 10 fold more efficient at catalysing the conversion of NADP⁺ to NADPH than the polypeptide in step a).

25. A method for converting NADP⁺ to NADPH or for converting NAD⁺ to NADH, comprising use of a polypeptide of claim 1.

26. An oxidoreductase process comprising (a) use of a polypeptide of claim 1; (b) the process of (a), wherein the oxidoreductase process regenerates NADH or NADPH; (c) the process of (a) or (b), wherein the oxidoreductase process causes insertion of an oxygen atom in a C—H or C—C bond, hydride delivery or reductive amination; or (d) the process of (a), (b) or (c), wherein the oxidoreductase process comprises a monooxygenation reaction, a Baeyer-Villiger oxidation, a ketone reduction or D-amino acid synthesis.

27-29. (canceled)

30. A recombinant expression vector comprising the polynucleotide of claim 20, wherein optionally the polynucleotide is operably linked to a promoter.

31. A transformed cell comprising a polynucleotide claim 20.

32. A method for the conversion of NADP⁺ to NADPH, or the conversion of NAD⁺ to NADH, comprising (a) providing a polypeptide of claim 1, and (b) adding it to NADP⁺ or NAD⁺.

33. An oxidoreductase process comprising (a) providing a polypeptide of claim 1, and (b) adding it to an oxidoreductase reaction mixture.


SEQUENCE LISTING (28 sequences)

SEQ ID NO: 1 - protein, 386 aa, Burkholderia sp.
MATVLCVLYPDPVDGYPPRYVRDAIPVITHYADGQTAPTPAGPPGFQPGELVGCVSGALGLRGYMEAHGHTLIVTSDKDSPDSEFERRLPEADVVISQPFWPAYLTAERIARAPKLKLALTAGIGSDHVDLDAAARARITVAEVTGSNSISVAEHVVMTTLALVRNYLPSHAIAQQGGWNIADCVSRSYDVEGMHFGTVGAGRIGLAVLRRLKPFGLQLHYTQRHRLDASIEQELGLTYHADAASLASAVDIVNLQIPLYPSTEHLFDAAMIARMKRGAYLINTARAKLVDRDAVVNALTSGHLAGYGGDVWFPQPAPADHPWRTMPFNGMTPHISGTSLSAQARYAAGTLEILQCWFDGKPIRNEYLIVDGGTLAGTGAQSYRLT

SEQ ID NO: 2 - protein, 386 aa, Burkholderia cenocepacia
MATVLCVLYPDPVDGYPPRYVRDTIPVITHYADGQTAPTPAGPPGFRPGELVGSVSGALGLRGYMEAHGHTLIVTSDKDGPDSEFERRLPEADVVISQPFWPAYLSAERIARAPKLKLALTAGIGSDHVDLDAAARARITVAEVTGSNSISVAEHVVMTTLALVRNYLPSHAVAQQGGWNIADCVSRSYDVEGMHFGTVGAGRIGLAVLRRLQPFGLQLHYTQRHRLDASIEQALALTYHADVASLASAVDIVNLQIPLYPSTEHLFDAAMIARMKRGAYLINTARAKLVDRDAVVNALTSGHLAGYGGDVWFPQPAPADHPWRTMPFNGMTPHISGTSLSAQARYAAGTLEILQCWFDGRPIRNEYLIVDGGTLAGTGAQSYRLT

SEQ ID NO: 3 - protein, 350 aa, Pseudomonas sp.
MAKVLCVLYDDPVDGYPKTYARDDLPKIDHYPGGQTLPTPKAIDFTPGQLLGSVSGELGLRKYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTDRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTR

SEQ ID NO: 4 - protein, 364 aa, Saccharomyces cerevisiae
MSKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYDYQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQN

SEQ ID NO: 5 - protein, 364 aa, Hordeum vulgare
MAAMWRAAARQLVDRAVGSRAAHTSAGSKKIVGVFYQAGEYADKNPNFVGCVEGALGIRDWLESKGHHYIVTDDKEGFNSELEKHIEDMHVLITTPFHPAYVTAEKIKKAKTPELLLTAGIGSDHIDLPAAAAAGLTVARVTGSNTVSVAEDELMRILILLRNFLPGYQQVVKGEWNVAGIAHRAYDLEGKTVGTVGAGRYGRLLLQRLKPFNCNLLYHDRLQINPELEKEIGAKFEEDLDAMLPKCDVVVINTPLTEKTRGMFNKEKIAKMKKGVIIVNNARGAIMDTQAVADACSSGHIAGYGGDVWFPQPAPKDHPWRYMPNHAMTPHISGTTIDAQLRYAAGVKDMLDRYFKGEEFPVEN

SEQ ID NO: 6 - protein, 364 aa, Candida methylica
MKIVLVLYDAGKHAADEEKLYGCTENKLGIANWLKDQGHELITTSDKEGETSELDKHIPDADIIITTPFHPAYITKERLDKAKNLKSVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 7 - protein, 350 aa, Hansenula polymorpha
MKVVLVLYDAGKHAQDEERLYGCTENALGIRDWLEKQGHDVVVTSDKEGQNSVLEKNISDADVIISTPFHPAYITKERIDKAKKLKLLVVAGVGSDHIDLDYINQSGRDISVLEVTGSNVVSVAEHVVMTMLVLVRNFVPAHEQIISGGWNVAEIAKDSFDIEGKVIATIGAGRIGYRVLERLVAFNPKELLYYDYQSLSKEAEEKVGARRVHDIKELVAQADIVTINCPLHAGSKGLVNAELLKHFKKGAWLVNTARGAICVAEDVAAAVKSGQLRGYGGDVWFPQPAPKDHPWRSMANKYGAGNAMTPHYSGSVIDAQVRYAQGTKNILESFFTQKFDYRPQDIILLN

SEQ ID NO: 8 - protein, 364 aa, Moraxella sp.
MAKVVCVLYDDPINGYPTSYARDDLPRIDKYPDGQTLPTPKAIDFTPGALLGSVSGELGLRKYLESQGHELVVTSSKDGPDSELEKHLHDAEVIISQPFWPAYLTAERIAKAPKLKLALTAGIGSDHVDLQAAIDNNITVAEVTYCNSNSVAEHVVMMVLGLVRNYIPSHDWARNGGWNIADCVARSYDVEGMHVGTVAAGRIGLRVLRLLAPFDMHLHYTDRHRLPEAVEKELNLTWHATREDMYGACDVVTLNCPLHPETEHMINDETLKLFKRGAYLVNTARGKLCDRDAIVRALESGRLAGYAGDVWFPQPAPNDHPWRTMPHNGMTPHISGTSLSAQTRYAAGTREILECYFEGRPIRD

SEQ ID NO: 9 - protein, 364 aa, Neurospora crassa
MVKVLAVLYDGGKHGEEVPELLGTIQNELGLRKWLEDQGHTLVTTCDKDGENSTFDKELEDAEIIITTPFHPGYLTAERLARAKKLKLAVTAGIGSDHVDLNAANKTNGGITVAEVTGSNVVSVAEHVLMTILVLVRNFVPAHEQIQEGRWDVAEAAKNEFDLEGKVVGTVGVGRIGERVLRRLKPFDCKELLYYDYQPLSAEKEAEIGCRRVADLEEMLAQCDVVTINCPLHEKTQGLFNKELISKMKKGSWLVNTARGAIVVKEDVAEALKSGHLRGYGGDVWFPQPAPQDHPLRYAKNPFGGGNAMVPHMSGTSLDAQKRYAAGTKAIIESYLSGKHDYRPEDLIVYGGDYATKSYGERER

SEQ ID NO: 10 - protein, 364 aa, Solanum tuberosum
MAMSRVASTAARAITSPSSLVFTRELQASPGPKKIVGVFYKANEYAEMNPNFLGCAENALGIREWLESKGHQYIVTPDKEGPDCELEKHIPDLHVLISTPFHPAYVTAERIKKAKNLQLLLTAGIGSDHVDLKAAAAAGLTVAEVTGSNTVSVAEDELMRILILVRNFLPGHHQVINGEWNVAAIAHRAYDLEGKTVGTVGAGRIGRLLLQRLKPFNCNLLYHDRLKMDSELENQIGAKFEEDLDKMLSKCDIVVINTPLTEKTKGMFDKERIAKLKKGVLIVNNARGAIMDTQAVVDACNSGHIAGYSGDVWYPQPAPKDHPWRYMPNQAMTPHISGTTIDAQLRYAAGTKDMLDRYFKGEDF

SEQ ID NO: 11 - protein, 401 aa, Thiobacillus sp.
MAKILCVLYDDPVDGYPKTYARDDLPKIDHYPGGQTLPTPKAIDFTPGQLLGSVSGELGLRKYLEANGHTFVVTSDKDGPDSVFEKELVDADVVISQPFWPAYLTPERIAKAKNLKLALTAGIGSDHVDLQSAIDRGITVAEVTYCNSISVAEHVVMMILGLVRNYIPSHDWARKGGWNIADCVEHSYDLEGMTVGSVAAGRIGLAVLRRLAPFDVKLHYTDRHRLPEAVEKELGLVWHDTREDMYPHCDVVTLNVPLHPETEHMINDETLKLFKRGAYIVNTARGKLADRDAIVRAIESGQLAGYAGDVWFPQPAPKDHPWRTMKWEGMTPHISGTSLSAQARYAAGTREILECFFEGRPIRDEYLIVQGGALAGTGAHSYSKGNATGGSEEAAKFKKAG

SEQ ID NO: 12 - protein, 403 aa, Legionella pneumophila
MFSQSQQKIVCVLYDDPKGGFPPNYARESIPELKQYPDGQSLPNPDSIDFIPGEMLGSVSGELGLRQFLESNGHQLVVTSDKDGSDSVFARELKEATVVISQPFWPAYLTRDRIESAPKLKLAITAGIGSDHVDLQAAMEHNITVCEVTYCNSISVAEHTVMMILALVRDFIPQYNTVIDGGWNIADCVSRSYDLEGMQVGCVAAGRIGLAVLRRLKPFAVKLHYTDRHRLPVQLEQELNLTYHPSVESMVKVCDVVSIHCPLHPETEYLFDERLIKQMKRGSYLINTARGRICDQHAVADALESGHLAGYAGDVWFPQPPAKNHPWRSMPNHAMTPHTSGTTLSAQARYAAGVREILECWLGNKPIREEYLIVSQGHLAGVGSHSYSAGNTTSGTEQAAELV

SEQ ID NO: 13 - protein, 364 aa, Candida boidinii
MKIVLVLYDAGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLVPFNPKELLYYDYQALPKDAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAQGTKNILESFFTGKFDYRPQDIILLNGEYVTKAYGKHDKK

SEQ ID NO: 14 - protein, 400 aa, Paracoccus sp.
MAKVVCVLYDDPVDGYPTSYARDSLPVIERYPDGQTLPTPKAIDFVPGSLLGSVSGELGLRNYLEAQGHELVVTSSKDGPDSELEKHLHDAEVVISQPFWPAYLTAERIAKAPKLKLALTAGIGSDHVDLQAAIDRGITVAEVTFCNSISVSEHVVMTALNLVRNYTPSHDWAVKGGWNIADCVTRSYDIEGMHVGTVAAGRIGLAVLRRFKPFGMHLHYTDRHRLPREVELELDLTWHESPKDMFPACDVVTLNCPLHPETEHMVNDETLKLFKRGAYLVNTARGKLCDRDAVARALESGQLAGYGGDVWFPQPAPQDHPWRTMPHNAMTPHISGTSLSAQARYAAGTREILECHFEGRPIRDEYLIVQGGSLAGVGAHSYSKGNATGGSEEAAKFKKA

SEQ ID NO: 15 - protein, 374 aa, Kluyveromyces lactis
MLRLTSARSIVSPLRKGAFGSIRTLATSVPETQKGVIFYENGGKLEYKDIPVPKPKPNEILINVKYSGVCHTDLHAWKGDWPLPTKLPLVGGHEGAGVVVAMGENVKGWNIGDFAGIKWLNGSCMSCEYCELSNESNCPDADLSGYTHDGSFQQYATADAVQAARIPKGTDLAEVAPILCAGVTVYKALKSANLKAGDWVAISGAAGGLGSLAVQYAKAMGYRVVGIDGGEEKGKLVKQLGGEAFVDFTKTKDMVAEIQEITNGGPHGVINVSVSEAAMNASTQFVRPTGTVVLVGLPAGAVIKSEVFSHVVKSINIKGSYVGNRADTREAINFFANGHVHSPIKVVGLSELPKVYELMEQGKILGRYVVDTSN

SEQ ID NO: 16 - protein, 371 aa, Shewanella sp.
MIIGVPTEIKNHEYRVGMVPSSVRELTIKGHVVYVQSDAGVGIGFTDQDYIDAGASILATAAEVFAKSDMIVKVKEPQAVERAMLRHDQILFTYLHLAPDLPQTEELITSGAVCIAYETVTDDRGGLPLLAPMSEVAGRMSIQAGARALEKSLGGRGMLLGGVPGVEPAKVVIIGGGMVGTNAAQMAVGMGADVVVLDRSIDALRRLNVQFGSAVKAIYSTADAIERHVLEADLVIGGVLVPGAAAPKLITRDMVKRMKPGSAIVDVAIDQGGCVETSHATTHQDPTYIVDDVVHYCVANMPGAVARTSTFALNNATLPYIIKLANQGYKQALLNDKHLLNGLNVMHGKVVCKEVAEALNLEFTEPKSLLA

SEQ ID NO: 17 - protein, 361 aa, Phormidium lapideum
MEIGVPKEIKNQEFRVGLSPSSVRTLVEAGHTVFIETQAGIGAGFADQDYVQAGAQVVPSAKDAWSREMVVKVKEPLPAEYDLMQKDQLLFTYLHLAAARELTEQLMRVGLTAIAYETVELPNRSLPLLTPMSIIAGRLSVQFGARFLERQQGGRGVLLGGVPGVKPGKVVILGGGVVGTEAAKMAVGLGAQVQIFDINVERLSYLETLFGSRVELLYSNSAEIETAVAEADLLIGAVLVPGRRAPILVPASLVEQMRTGSVIVDVAVDQGGCVETLHPTSHTQPTYEVFGVVHYGVPNMPGAVPWTATQALNNSTLPYVVKLANQGLKALETDDALAKGLNVQAHRLVHPAVQQVFPDLA

SEQ ID NO: 18 - protein, 363 aa, Pichia stipitis
MTANPSLVLNKIDDISFETYDAPEISEPTDVLVQVKKTGICGSDIHFYAHGRIGNFVLTKPMVLGHESAGTVVQVGKGVTSLKVGDNVAIEPGIPSRFSDEYKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVDIFDNKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPE

SEQ ID NO: 19 - protein, 336 aa, Pseudomonas stutzeri
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAAC

SEQ ID NO: 20 - DNA, 29 bp, Artificial Sequence
  Description of Artificial Sequence Synthetic primer
cgatgtccatatggctaccgtcctgtgcg

SEQ ID NO: 21 - DNA, 27 bp, Artificial Sequence
  Description of Artificial Sequence Synthetic primer
cacctcgagcgtcagccggtacgactg

SEQ ID NO: 22 - DNA, 29 bp, Artificial Sequence
  Description of Artificial Sequence Synthetic primer
cgatgtccatatggccaccgtcctgtgcg

SEQ ID NO: 23 - DNA, 27 bp, Artificial Sequence
  Description of Artificial Sequence Synthetic primer
cacctcgagtgtcagccggtacgactg

SEQ ID NO: 24 - DNA, 32 bp, Artificial Sequence
  Description of Artificial Sequence Synthetic oligonucleotide
gaattattatactaccagtatcaagctttacc

SEQ ID NO: 25 - DNA, 32 bp, Artificial Sequence
  Description of Artificial Sequence Synthetic oligonucleotide
ggtaaagcttgatactggtagtataataattc

SEQ ID NO: 26 - DNA, 29 bp, Artificial Sequence
  Description of Artificial Sequence Synthetic oligonucleotide
gctgcactacacggatcgccaccggctcg

SEQ ID NO: 27 - DNA, 29 bp, Artificial Sequence
  Description of Artificial Sequence Synthetic oligonucleotide
cgagccggtggcgatccgtgtagtgcagc

SEQ ID NO: 28 - protein, 8 aa, Artificial Sequence
  Description of Artificial Sequence Synthetic peptide
LEHHHHHH